(19) United States
(12) Patent Application Publication
(10) Pub. No.: US 2023/0421996 A1
(43) Pub. Date: Dec. 28, 2023

(54) SYSTEM AND METHOD FOR DETERMINING THE LOCATION OF PERSONS

(71) Applicant: Paimcos Pty Ltd, Ultimo (AU)

(72) Inventor: Adrian Iordachescu, Ultimo (AU)

(21) Appl. No.: 18/250,424

(22) PCT Filed: Aug. 25, 2021
(86) PCT No.: PCT/AU2021/050966

§ 371 Date: Apr. 25, 2023

(30) Foreign Application Priority Data
Oct. 26, 2020 (AU) 2020903871

(51) Int. Cl. H04W 4/029; G01S 19/14; G01C 5/06

(52) U.S. Cl. CPC H04W 4/029; G01S 19/14; G01C 5/06

(57) ABSTRACT

There is provided a method for determining whether a person is located within an acceptable locality, the person being associated with a mobile communication device. The method comprises sending a first location confirmation message to the mobile communication device over a communication means, the location confirmation message comprising a reference to location confirmation code, receiving a request from the mobile communication device to access the location confirmation code, and sending, to the mobile communication device, the location confirmation code. The method further comprises receiving, from the mobile communication device, location data indicative of a location of the mobile communication device, and determining, based on the location data, whether the location of the mobile communication device is within the acceptable locality.

### CROSS-REFERENCE TO RELATED APPLICATIONS

The present application is a 371 of International Patent Application No. PCT/AU2021/050966, filed on 25 Aug. 2021, which claims priority from Australian Provisional Patent Application No 2020903871 filed on 26 Oct. 2020, the contents of both being incorporated herein by reference in their entirety.

### TECHNICAL FIELD

This disclosure relates to systems and methods for confirm the location of a person of interest, and identifying attempted boundary crossings by a restricted person.

### BACKGROUND

A person may be required to remain within a locality for purposes such as the safety and security of the person or others, legal or correctional purposes, or medical purposes such as quarantine management in the event of an epidemic or pandemic.

For example, in the event of an epidemic or pandemic, it may be necessary to instigate quarantine and isolation requirements to individuals. These requirements may specify that an individual is to remain within a defined location, such as a residence, for a defined period. Additionally, in the event of an epidemic or pandemic, it may be necessary to instigate movement restrictions, such that certain persons are restricted from entering defined regions, facilities or venues, on medical grounds.

Accordingly, there is a desire to determine, with a level of confidence, the location of a person who is subject to a locality requirement or movement restriction.

For applications that seek to determine the individual locations of a large number of persons in a population, it is desirable to utilise a location determination process that is efficient and can be readily performed without the use of highly specialised technology.

Additionally, for some applications, it is desirable to minimise the invasiveness of the location determination process, such that persons are not greatly inconvenienced by the process.

Any discussion of the prior art throughout the specification should in no way be considered as an admission that such prior art is widely known or forms part if the common general knowledge in the field.

### SUMMARY

The present disclosure provides a system and method for remotely determining the location of a person of interest. The present disclosure further provides a system and method for identifying when a person of interest is attempting to cross a boundary into a restricted region.

According to a first aspect of the present disclosure, there is provided a system for determining whether a person is located within an acceptable locality, the person being associated with a mobile communication device. The system comprises a server configured to, send a first location confirmation message to the mobile communication device over a communication means, the location confirmation message comprising a reference to location confirmation code, receive a request from the mobile communication device to access the location confirmation code, send, to the mobile communication device, the location confirmation code, receive, from the mobile communication device, location data indicative of a location of the mobile communication device, determine, based on the location data, whether the location of the mobile communication device is within the acceptable locality.

According to a second aspect of the present disclosure, there is provided a method for determining whether a person is located within an acceptable locality, the person being associated with a mobile communication device. The method comprises sending a first location confirmation message to the mobile communication device over a communication means, the location confirmation message comprising a reference to location confirmation code, receiving a request from the mobile communication device to access the location confirmation code, sending, to the mobile communication device, the location confirmation code, receiving, from the mobile communication device, location data indicative of a location of the mobile communication device, determining, based on the location data, whether the location of the mobile communication device is within the acceptable locality.

The method may further comprise receiving, from the mobile communication device, first biometric data of a first biometric attribute type, obtaining, first reference biometric data, of a first biometric attribute type, associated with the person, and determining whether the first biometric data matches the first reference biometric data.

The method may further comprise sending a second location confirmation message to the mobile communication device over a communication means.

The method may further comprise sending a second location confirmation message to the mobile communication device over a communication means, receiving, from the mobile communication device, second biometric data of a second biometric attribute type, obtaining, second reference biometric data, of a second biometric attribute type, associated with the person, and determining whether the second biometric data matches the second reference biometric data, wherein the first biometric attribute type is different to the second biometric attribute type.

The method may further comprise sending an request to initialise message to the mobile communication device over a communication means, the request to initialise message comprising shared-knowledge information and contact information.

Each of the first location confirmation message and the second location confirmation message may include an authentication keyword, and the authentication key word included in the first location confirmation message may be the same as the authentication key word included in the second location confirmation message.

Each of the first location confirmation message and the second location confirmation message may include a sequence number, and the sequence number included in the second location confirmation message may be equal to the sequence number included in the first location confirmation message incremented by one.

The method may further comprise determining a period of time that has elapsed since performing the step of sending the first location confirmation message to the mobile communication device, and in response to the period of time being greater than a threshold period of time, sending a second location confirmation message to the mobile communication device over a communication means.

The method may further comprise adjusting the threshold period of time based on a number of restricted persons in the set of restricted persons associated with a name that is equal to the name associated with the person.

The method may further comprise determining a period of time based on a monitoring intensity value associated with the person, and sending a second location confirmation message to the mobile communication device the period of time after sending the first location confirmation message to the mobile communication device.

The method may further comprise adjusting the monitoring intensity associated with the person based on a number of restricted persons in the set of restricted persons associated with a name that is equal to the name associated with the person.

The method may further comprise, in response to the location of the mobile communication device being outside the acceptable locality, outputting an alert message. In one embodiment the location data comprises GPS coordinates. In one embodiment the location data comprises a barometric pressure measurement, or indication thereof.

According to a third aspect of the present disclosure, there is provided a device for determining whether a checkpoint user is permitted to cross a boundary, the boundary comprising a checkpoint, and the checkpoint user being associated with a name. The device comprises a memory store comprising a set of restricted names, each restricted name of the set of restricted names being associated with one restricted person of a set of restricted persons, and a server. The server is configured to receive a name from a checkpoint management device, determine whether the name is the same as one or more of the restricted names in the set of restricted names, and in response to the name not being the same as any of the restricted names in the set of restricted names, send a message to the checkpoint management device comprising a positive permission outcome.

The device may, in response to the name being the same as one of the restricted names in the set of restricted names, send a message to the checkpoint management device comprising a negative permission outcome.

The server may be further configured to determine a candidate set of restricted persons, the candidate set being a subset of the set of restricted persons, wherein the candidate set is determined based on the acceptable locality associated with each of the restricted persons of the set of restricted persons.

In response to the name being the same as one or more of the restricted names in the set of restricted names, the device may send a message to the checkpoint management device requesting at least one biometric attribute of the checkpoint user.

The server may be further configured to determine a subset of the set of restricted persons, each of the subset of restricted persons being associated with a name equal to the received name, receive, from the checkpoint management device, at least one biometric attribute of the checkpoint user, obtain at least one biometric attribute of one or more restricted persons in the set of restricted persons, compare the at least one biometric attribute of the checkpoint user to the at least one biometric attribute of the one or more restricted persons in the set of restricted persons, and in response to the at least one biometric attribute of the checkpoint user matching the at least one biometric attribute of at least one of the restricted persons, send a message to the checkpoint management device comprising a negative permission outcome.

The at least one biometric attribute of the checkpoint user may comprise a fingerprint, a retinal scan, or an image comprising the checkpoint user's face.

According to a fourth aspect of the present disclosure, there is provided a device for managing a boundary checkpoint. The device comprises a first input for receiving input from the checkpoint user, a second input for obtaining biometric attributes of a checkpoint user, an communication means for communicating with boundary crossing management server, and a processor. The processor is configured to receive, via the first input, from the checkpoint user, an indication of a name, send, to the boundary crossing management server, the indication of the name, receive, from the boundary crossing management server, a permission outcome for the checkpoint user to cross the checkpoint, in response to the permission outcome being positive, allow the checkpoint user to cross the checkpoint, and in response to the permission outcome being negative, inhibit the checkpoint user from crossing the checkpoint.

The device may further comprise a output operably coupled to a checkpoint barrier. The processor may be further configured to, in response to the permission outcome being negative, activate the checkpoint barrier to inhibit the checkpoint user from crossing the checkpoint.

The processor may be further configured to receive, from the boundary crossing management server, a request to obtain an biometric attribute of the checkpoint user, obtain, via the second input the biometric attribute of the checkpoint user, and send, to the boundary crossing management server, the biometric attribute of the checkpoint user.

### BRIEF DESCRIPTION OF DRAWINGS

The embodiments of the disclosure will now be described with reference to the accompanying drawings, in which:

FIG. 1 is a system diagram illustrating the components of a system for confirming the location of a POI, according to an embodiment;

FIG. 2 is a system diagram illustrating the communication protocols and application programming interfaces used within the system of FIG. 1, according to an embodiment.

FIG. 3 is a block diagram illustrating components of the server, according to an embodiment;

FIG. 4 is a window of a user interface of the location confirmation system, according to an embodiment;

FIG. 5 illustrates a scenario in which the server is unable to confirm the location of a mobile communication device, according to an embodiment;

FIG. 6 illustrates another scenario in which the server is unable to confirm the location of a mobile communication device, according to an embodiment;

FIG. 7 is a flowchart illustrating the location confirmation process, according to an embodiment;

FIG. 8 is a flowchart illustrating the process for performing a location search of a POI, according to an embodiment;

FIG. 9 illustrates privacy protection points for location confirmation system, according to an embodiment;

FIG. 10 is a flowchart illustrating a process for registering a POI with the location confirmation process and for initialising a location confirmation message sequence, according to an embodiment;

FIG. 11 is a flowchart illustrating a process for authenticating a message of a sequence of location confirmation messages, according to an embodiment;

FIG. 12 illustrates a display, or part thereof, of mobile communication device, according to an embodiment;

FIG. 13 illustrates a display, or part thereof, of mobile communication device, according to an embodiment;

FIG. 14 illustrates a display, or part thereof, of mobile communication device, according to an embodiment;

FIG. 15 illustrates a display, or part thereof, of mobile communication device, according to an embodiment;

FIG. 16 illustrates a display, or part thereof, of mobile communication device, according to an embodiment;

FIG. 17 illustrates a display, or part thereof, of mobile communication device, according to an embodiment;

FIG. 18 is a flowchart illustrating a process for re-establishing channel trust between the server and the mobile communication device, according to an embodiment;

FIG. 19 is a system diagram illustrating the components of a system in which the location confirmation process includes the steps of obtaining and verifying biometric data, according to an embodiment;

FIG. 20 is a flowchart illustrating a process for collecting a reference set of biometric data to be associated with the POI, according to an embodiment;

FIG. 21 is a flowchart illustrating a process for collecting biometric data, according to an embodiment;

FIG. 22 is a flowchart illustrating a process for comparing biometric data with reference biometric data, according to an embodiment;

FIG. 23 illustrates a component of the user interface of the location confirmation process, according to an embodiment;

FIG. 24 illustrates boundary crossing management at a checkpoint, according to an embodiment;

FIG. 25 is a flowchart illustrating the checkpoint management process, according to an embodiment;

FIG. 26 is a diagram illustrating a map of a region, according to an embodiment;

FIG. 27 is a flowchart illustrating the green path validation process, according to an embodiment;

FIG. 28 is a flowchart illustrating the amber path validation process, according to an embodiment;

FIG. 29 illustrates the change in the area represented by a POI's MDT, as the MDT increases over time, according to an embodiment;

FIG. 30 illustrates areas in which POIs could have travelled at a point in time, according to an embodiment;

FIG. 31 illustrates areas in which POIs could have travelled at a point in time, according to an embodiment;

FIG. 32 displays a monitoring profile for a POI, according to an embodiment;

FIG. 33 displays a monitoring profile for a POI, according to an embodiment;

FIG. 34 is a flowchart illustrating the process for optimisation of a monitoring profile for a POI, according to an embodiment; and

FIG. 35 illustrates a display, or part thereof, of mobile communication device, according to an embodiment.

### DESCRIPTION OF EMBODIMENTS

In the event of a pandemic or epidemic, it may be desirable to identify persons that are required to quarantine or isolate for medical reasons. Such persons are called a person of interest (POI). A POI may be required, by some Decision Making Authority (DMA), to remain at, or acceptably near to, a required location. The required location may be the POI's place of residence, a medical facility, a quarantine facility or other location.

In one embodiment, location monitoring is managed by a Decision Making Authority (DMA), such as a pandemic task force. The DMA utilises a location confirmation system to periodically determine whether a POI is located at, or acceptably near to, the required location associated with that person.

The present disclosure provides systems and methods for confirming that a person of interest (POI) is located in a designated locality in fast and simple way. A server, managed by the DMA, uses a Mobile Network Messaging protocol to communicate a resource link to a mobile communication device associated with the POI. The resource associated with the resource link enables the mobile communication device to obtain the Global Positioning System (GPS) coordinates of the mobile communication device and provide the coordinates to the server managed by the DMA. As a result, the server obtains the location of the mobile communication device associated with the POI. Additional techniques, as described below, provide verification that the POI is located in close proximity to the mobile communication device.

### Mobile Network Messaging (MNM)

A Mobile Network Messaging (MNM) is a wireless information transferring mechanism, that allows communication from a server managed by the DMA to a mobile communication device operated by the POI.

In some embodiments, it may be desirable that the MNM is available across all mobile networks. Additionally, for some embodiments, it is desirable that the MNM technology is publicly available i.e. not restricted to users of a particular platform or application.

An example MNM is Short Message Service (SMS). SMS does not need mobile data or Wi-Fi to send and receive messages. This makes it a convenient option when there is limited internet connectivity. Embodiments of the present disclosure use the SMS communication protocol for communicating between the location confirmation server and a mobile communication device of a POI; however, the functionality presented herein may be implemented via any wireless information transferring mechanism, that allows communication from a server managed by the DMA to a mobile communication device operated by the POI.

Other communication protocols that may be used include Multimedia Messaging Server (MMS) and Rich Communication Services (RCS). Communication applications such as Facebook Messenger, WhatsApp, iMessage and WeChat may be used, however these communication application are private, and therefore may require a login for access.

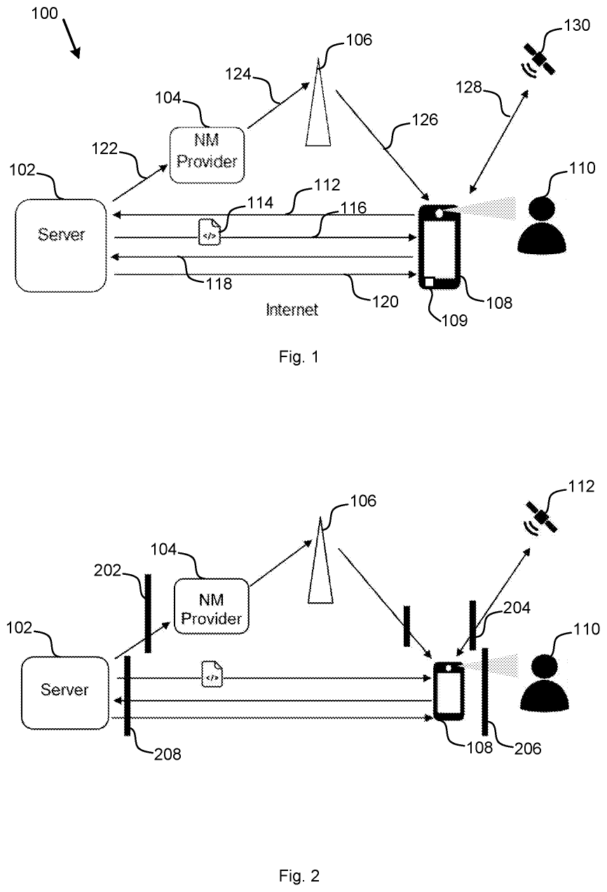

### FIG. 1—System Architecture

FIG. 1 is a system diagram illustrating the components of a system 100 for confirming the location of a POI, according to an embodiment. The system 100 comprises a location confirmation system server 102 in communication with a network messaging provider 104. In the embodiment illustrated in FIG. 1, provider 104 is a Simple Messaging Service (SMS) provider. As detailed above, in other embodiments, the provider 104 may be MNM provider other than an SMS provider.

The SMS provider 104 is in communication with at least one mobile phone tower 106. The mobile phone tower 106 is configured to send SMS messages to a mobile device 108 associated with a person of interest (POI) 110. The mobile device 108 is associated with an identifier which uniquely identifies the mobile device 108 or an application executing thereon. In the embodiment illustrated in FIG. 1, the mobile device identifier is the mobile phone number of the mobile communication device 108. In another embodiment, the mobile device identifier maybe a username used to login in to an application executing on the mobile device.

In operation, the server 102 sends a request 122 to the SMS provider 104. The request 122 comprises the mobile device identifier (e.g. a mobile phone number) and a message body. The message body comprises text and a URL.

In response to receiving the request 122, the SMS provider 104 sends an SMS comprising the message body to the mobile device 108 via the mobile tower 106. The mobile device 108 receives the SMS and provides a notification of receipt of the SMS to the POI 110 associated with the mobile device 108. The notification may be in the form or an audible, visible or tactile alert.

In response to receiving the notification, the POI 110 clicks on the URL included within the SMS. The URL provides a resource locator to a webpage hosted by the server 102. Clicking on the URL activates a browser application on the mobile device 108. The browser application downloads the webpage source code 114 from the server 102, by sending a HTTP request 112 to the server 102 and receiving a HTTP response from the server 102. The webpage source code 114 comprises geo-location determination code (or a referent thereto). The geo-location determination code is executable by the browser application on the mobile device 108. The browser application on the mobile device 108 executes the geo-location determination code. The geo-location determination code determines the geo-location of the mobile device 108, at least in part by requesting and receiving 128 geo-location coordinates from a base station 130. In some embodiments, the geo-location determination code determines additional aspects of the geo-location coordinates of the mobile device 108 via sensors associated with the mobile device 108, such as a barometric sensor 109.

It is noted that communication 112 may comprises a plurality of communication instances. Additionally, communication 114 may comprise a plurality of communication instances, in order to download the geo-location source code to the mobile communication device 108.

The browser application on the mobile device 108 sends 118 the geo-location coordinates to the server 102. The geo-location coordinates are otherwise known as location data, or geo-location data. The location data may comprise GPS coordinates, an indication of barometric pressure, or a combination thereof.

The server 102 determines whether the geo-location coordinates indicate a location that is within an acceptable boundary. In response to the server 102 determining that the geo-location coordinates indicate a location that is within an acceptable boundary, the server 102 sends 120 a confirmation message to the mobile device 108. The mobile device 108 may then provide an audible, visual or tactile notification to the POI that a confirmation message has been received from the server 102.

In one embodiment, the geo-location code is a JavaScript component of the webpage 114, that is configured to access the GPS sensor of the mobile communication device 108

### Communicating GPS Location to Server

In communication 118, the mobile device 108 communicates the location of the mobile communication device, as determined by the GPS location, to the server 102. In the embodiment illustrated in FIG. 1, the mobile device 108 communicates 118 over the Internet; however, in other embodiments, the mobile device 108 communicates 118 over SMS text or other data connection.

### Mobile Communication Device

The mobile communication device 108 may be a mobile phone, or other mobile communication device. The mobile communication device 108 is configured to receive messages, comprising at least text and a URL. Furthermore, the mobile communication device is configured to, in response to input provided by an operator, download an execute the resource indicated by the URL. The URL will indicate a resource available over the internet. Accordingly, the mobile communication device 108 has internet access capability. The mobile communication device 108 is configured with a location sensor, or access thereto. The location sensor may comprise a GPS sensor. The location sensor may further comprise a barometric sensor.

### GPS Receiver

A GPS receiver receives the information from all available satellites and calculates the GPS receiver's exact location by comparing the time that a signal was transmitted by the satellite to the time the receiver receives the signal. This provides the distance that the satellite is from the receiver. By using this difference from several satellites, the GPS receiver is able to determine the receiver's position with a high degree of accuracy and display on a map or chart.

Most modern cell phones come with a built-in GPS receiver in the phone. This receiver works similarly to other commercial-grade GPS receivers, in that it uses the difference in time of transmission and receipt of the GPS signal from a satellite to determine the distance to the satellite from the phone. If two satellites are visible by the phone, then a 2D position can be calculated. The greater the number of satellites visible to the phone, the more accurate the position will be. As a result, if the location services of a phone are turned on, the cell phone will be calculating the device's position in near real time or real-time depending on the phone.

### Geo-Location Code

The server 102 sends a location confirmation message, via MNM, to the mobile communication device 108. As noted above, the location confirmation message may be sent via a MNM protocol. In the embodiment illustrated in FIG. 1, the server 102 sends the location confirmation message via SMS.

The location confirmation message contains the embedded link in the form of a URL. Activation of the link, via clicking or touch, triggers a browser on the mobile device 108 to download a webpage from the server. The webpage includes geo-location code, which runs locally on the mobile communication device 108 to access the device's GPS sensor, to determine the current location of the mobile communication device 108. Then geo-location code instructs the mobile communication device 108 to transfer 118 the longitude and latitude location coordinates to the server 102 via the Internet. The longitude and latitude location coordinates form geo-location coordinates.

### Location Height

In some situations, it may be desirable to determine the height of the POI's location. This may be relevant if the required location of the person is within a multi-storied structure, such as a high-rise apartment. In one embodiment, the geo-location code determines the altitude of the mobile device 108 using the device's GPS sensor. In another embodiment, the mobile device 108 comprises a barometric sensor and the geo-location code executing on the mobile device 108 interacts with the barometric sensor to obtain a barometric pressure measurement, or indication thereof. The barometric pressure measurement can provide an indication of the height of the mobile device relative to the surface of the earth.

The geo-location code, executing on the mobile device 108, provides the barometric pressure measurement, or an indication thereof, to the server 102 via the Internet, as part of the geo-location coordinates.

### Capabilities of the Mobile Device

To operate in accordance with one embodiment of the present disclosure, the mobile communication device 108 has Internet connectivity. Internet connectivity can be provided via the mobile network (e.g. connectivity with at least one mobile tower with data access) or via other means such as Wi-Fi. The mobile communication device 108 also includes a browser application, a GPS sensor and permissions are enabled for the browser application to access the GPS sensor. Such permission can be granted by the operator of the mobile communication device 108.

### Configuration Settings

The server 102 maintains a set of configuration settings for each of the persons of interest managed by the server. In one embodiment, a set of configuration settings is shared for a plurality of POIs. In one embodiment, the configuration settings include: an identification for the mobile communication device (e.g. a mobile phone number); GPS coordinates of the required location for the POI; and the POI's name. In one embodiment, the configuration settings further include a height indication of the required location for the POI. The height indication may be in the form of a barometric pressure measurement.

The configuration settings may also include an indication of location tolerance. Location tolerance indicates the distance from the required location that would still be considered acceptable by the location confirmation system. Location tolerance is set on a case by case basis.

In one example, the location tolerance is set to represent an acceptable area that is the average size of a home, such as a radius of 25 to 100 metres from the required location. In another example, in which the POI is required to locate at a rural location, the location tolerance may be larger. A default location tolerance of 50 metres can be set in the configuration settings.

The configuration settings for a POI may also include an indication of one or more monitoring intensities. Monitoring intensity specifies a range for the number of location confirmation checks that are performed in a given timeframe. A default value could be 6-8 location confirmation messages per 12 hours. An intense monitoring profile could double that to 12-16 location confirmation messages per 12 hours. In one embodiment, a the monitoring intensity is set to a range, rather than a specific value to avoid making the monitoring predictable.

The configuration settings for a POI may also include an indication of maximum check interval (MCI). MCI represents the maximum time period between 2 location confirmation checks. The unit of measure for MCI is hour (h). As location confirmation checks may be performed periodically, with random or pseudo-random intervals, the MCI ensures that an extended period between location confirmation checks does not occur. In one embodiment, the MCI is set to a value that is double the time interval between location confirmation checks, if the location confirmation checks were equally distributed over a check period. For example, for a Monitoring Intensity of 6-8 messages per 12 hours, e.g. 1 message every 1.5-2 hours, MCI could be set to 4 hours.

The configuration settings for a POI may also include an indication of monitoring profile. The monitoring profile for a POI indicates time spans over a given time period, (say 24 hours or even a week) in which a particular monitoring intensity will apply. If this information is not present a default monitoring profile will be applied. An example monitoring profile is presented below:
12:00 am-6:00 am—low intensity 1-2 messages per 12 hours
6:01 am-5:30 pm—standard intensity 6-8 messages per 12 hours
5:31 pm-9:30 pm—increased intensity 1-2 messages per 1 hour
9:31 pm-11:59 pm—low intensity 1-2 messages per 12 hours

A monitoring profile can be configured for each POI, based on characteristics or circumstances of the POI. In the example above, the increased monitoring intensity in the time span of 5:31 pm to 9:30 pm is desirable as there is an increased likelihood that this POI will seek to move from the POI's required location in this evening period.

### Acceptable Location Boundary

After receiving 118 the geo-location coordinates from the mobile communication device 108, the server 102 determines whether the location of the mobile communication device 108 is within an area defined by the POI's acceptable location boundary. The area defined by the POI's acceptable location boundary is the POI's acceptable location. In one embodiment, the acceptable location boundary for a POI is defined by a circumference of a circle centred on the required location of the POI, with a radius defined by the location tolerance for the POI. In another embodiment, the acceptable location boundary is defined by a perimeter which is defined in terms of GPS coordinates. The perimeter may be irregular in shape. In one embodiment, the acceptable location boundary for a POI is defined by a height indicator, or height range. The height indicator or height range may be defined with reference to one or more barometric pressure values.

The acceptable boundary can be configured for each POI. The accuracy of GPS coordinates is good enough to set boundaries down to a 5-6 m radius which would correspond to a house/unit. In general, a radius of 50 to 100 m may be more practical.

### Applications of the Location Confirmation System

Embodiments of the location confirmation system, as performed by server 102, may find application in quarantine monitoring, boundary crossing management and search and rescue situations. When used for location monitoring, the system is used to monitor if a POI is located in a designated area for the agreed duration.

In this scenario the system will monitor via periodic, random SMS messages that will need to be confirmed by the POI. The system has different parameters that control the monitoring in terms of intensity, area size and schedules, for example day versus night. In this type of scenarios, the interaction between the DMA and the POI is on-going for an agreed period of time and subject to compliance frameworks.

### Boundary Crossing Management

Embodiments of the location confirmation system find application in boundary crossing management (otherwise called ‘hotspot management’). Boundary crossing management seeks to identify the restricted entry or exit of a person of interest from restricted region. A restricted region may be a regional borders or a venue or facility. Boundary crossing management is described in depth below.

### Search and Rescue

Embodiments of the location confirmation system find application in search and rescue operations. In search and rescue operations a member of a search and rescue team can utilise the location confirmation system to confirm the location of a person by sending a location confirmation message to a mobile communication device associated with the person. The rescue worker only needs to know the mobile phone number of the person to be rescued to be able to locate the person to be rescued. In a search and rescue operation, the person to be rescued is likely to be cooperative with the location confirmation system, and will follow the instructions in the MNM to allow their location to be immediately presented to the rescue worker. In a search and rescue situation, the interaction between the location confirmation system and the POI is on an ad-hoc basis, rather than a continuous periodic location monitoring.

### FIG. 2—Communication Protocols

FIG. 2 is a system diagram illustrating the communication protocols and application programming interfaces (API) used within system 100, according to an embodiment. The server 102 communicates with the NM provider 104 via an Internet connection 202. The server 102 communicates with the mobile communication device 108 via an Internet connection 208. An API 204 enables communication between the code executing on the mobile communication device and the GPS satellite 112, and an API 206 enables code executing on the mobile communication device to access the device's camera.

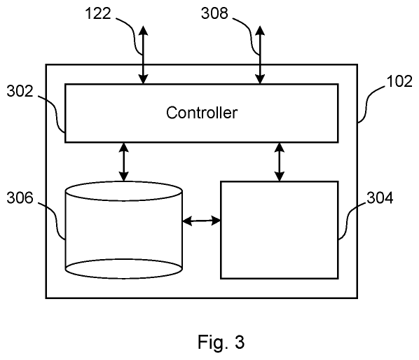

### FIG. 3—Server Architecture

FIG. 3 is a block diagram illustrating components of the server 102, according to an embodiment. Server 102 comprises a controller 302 which is a processing unit configured to perform the location confirmation process and execute the location confirmation user interface. The controller 302 is in communication with one or more SMS providers 104 via communication connection 122. The controller 302 is in communication with one or more mobile communication devices via communication connection 308. Communication connection 308 may be an Internet interface.

Server 102 further comprises a memory store 306, in which details of the POIs and their associated mobile communication devices are stored. Memory store 306 also stores configuration data for the location confirmation process for each POI, and log data recording the location confirmation history for each POI.

For brevity, embodiments described within this disclosure comprise the location confirmation system implemented on a single computation device referred to as server 102. However, in other embodiments, components of the location confirmation system may be implemented across a plurality of computation components, such as a plurality of servers. The plurality of computation components may form a distributed system, or may be a set of individual systems capable of operating in parallel. For example, location confirmation processes and boundary crossing management processes (described below) may be distributed across a plurality of servers based on regional needs or processing capabilities of the servers.

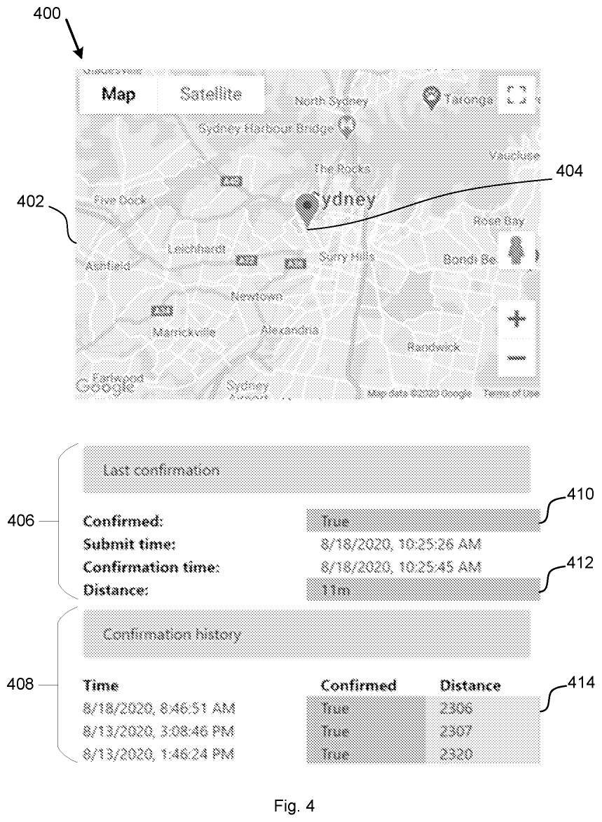

### FIG. 4—Location Confirmation History

FIG. 4 is an example window 400 of a user interface of the location confirmation system, showing the location confirmation history for a POI, according to an embodiment. The map 402 at the top of the window illustrates a geographic region. Geo-location point 404 indicates the location within the geographic region at which the POI 110 is required to be located in accordance with requirements placed upon the POI.

Section 406 of the window 400 shows details of the last location confirmation operation performed for the POI 110. A location confirmation operation comprises a location confirmation request sent from the server 102 to the mobile communication device 108 associated with a POI 110. Preferably, a location confirmation operation further comprises a location confirmation reply sent from the mobile communication device 108 and received by the server 102; however, it is noted that a confirmation reply may not be provided in response to a confirmation request being sent to the mobile communication device 108.

Section 406 shows details of the last confirmation request sent to the mobile communication device 108 associated with the POI 110, and the associated confirmation reply received from the mobile communication device. Item 410 indicates that a confirmation message was received from the mobile communication device 108. Furthermore, section 406 indicates that the last confirmation request was sent at time 10:25:26 AM, and the server 102 received a confirmation reply at time 10:25:45 AM. Item 412 indicates that the geo-location coordinates provided within the confirmation reply from the mobile communication device 108 indicated that the mobile communication device 108 was 11 metres from the POI's required location 404.

In the embodiment illustrated in FIG. 4, the acceptable boundary for the location of the mobile communication device 108 is within 50 metres of the POI's required location point 404. Accordingly, the location of the mobile communication device 108 11 metres from the required location point 404 is within the acceptable boundary.

Section 408 of window 400 shows details of three previous location confirmation requests. Notably, the coordinates provided within the confirmation reply from the mobile communication device 108, for each of the three previous location confirmation requests, indicates that the mobile communication device 108 is 2306, 2307, or 2320 metres, respectively, from the required location point 404. Accordingly, the location of the mobile communication device 108 at the time of the three previous location confirmation requests was outside the acceptable boundary.

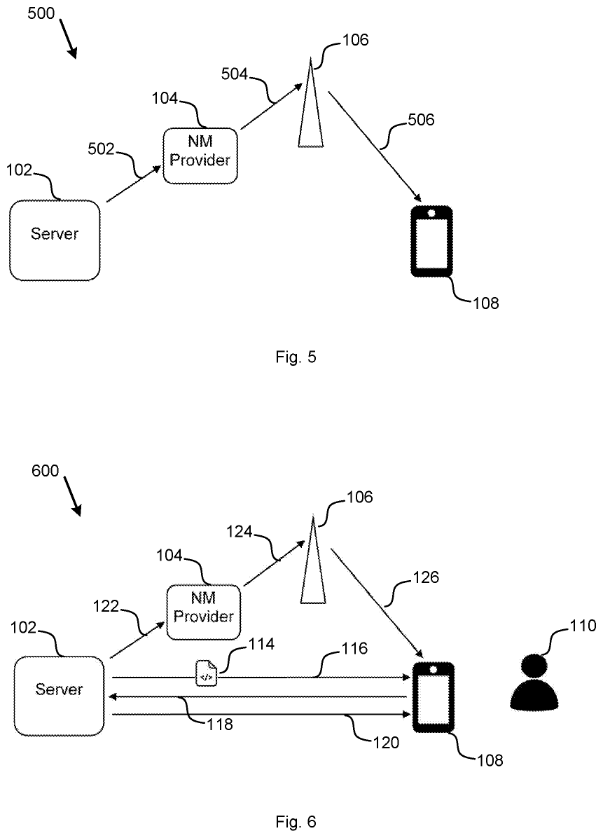

### FIGS. 5 and 6—Unsuccessful Confirmation

FIGS. 5 and 6 illustrate two a scenarios in which a server is unable to confirm the location of a mobile communication device within a defined time interval, according to an embodiment.

FIG. 5 illustrates a scenario 500 in which the server 102 is unable to confirm the location of a mobile communication device 108, according to an embodiment. The server 102 is unable to confirm the location of the mobile communication device 108 because the POI 110 does not access the URL provided in the location confirmation request message sent to the mobile communication device.

The server 102 sends 502 the location confirmation request, including a URL, to the SMS provider 104. The SMS provider 104 sends the location confirmation request via the mobile tower 106, to the mobile communication device 108. The mobile communication device 108 provides an alert indicating the receipt of the location confirmation request. The POI fails to activate the URL link provided in the location confirmation request, and after a defined period, the server 102 records the timeout of the location confirmation request in a location confirmation log. The server 102 may re-send the location confirmation request after a period of time.

### FIG. 6—Unsuccessful Confirmation Due to GPS Access Permissions

FIG. 6 illustrates a scenario 600 in which the server 102 is unable to confirm the location of a mobile communication device 108 because access has not been granted to the web browser to access the GPS sensor, according to an embodiment.

In this situation, the location confirmation system will send an alert to the decision making authority (DMA). The DMA may make contact with the POI via a phone call or other means to assist the POI to enable access to the GPS sensor of the mobile communication device.

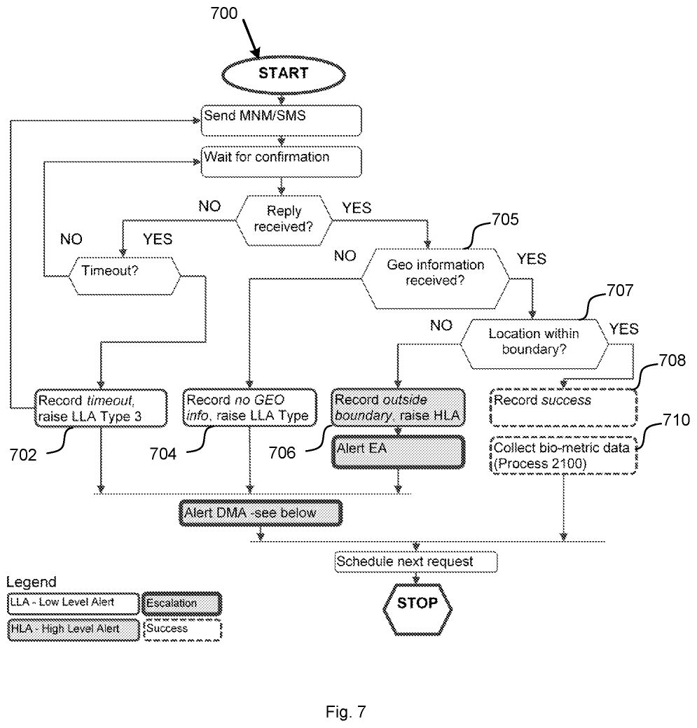

### FIG. 7—Location Confirmation

FIG. 7 is a flowchart illustrating the location confirmation process 700, as performed by server 102, for confirming the location of a POI 108, according to an embodiment. The server 102 executes process 700 iteratively, such that subsequent iterations of process 700 are performed sometime after completion of an iteration of process 700.

A process to perform a location confirmation can result in one of four outcomes, being: a timeout outcome; no geographic information outcome; location outside acceptable boundary outcome; or location within acceptable boundary outcome (otherwise called a success outcome).

A timeout outcome 702 occurs when the server 102 does not receive a request 112 to download the webpage 114 comprising the geo-location code, from the mobile communication device within a defined time period. This outcome may occur if the operator of the mobile communication device 108 (presumably, the POI 110) does not activate the URL link provided in the location request message. A timeout outcome will be recorded as a Low-Level Alert (LLA) and it will be escalated to DMA.

Depending on the configuration settings associated with the POI 108, a timeout outcome may not necessarily be a breach of the monitoring conditions. The POI can be contacted directly, for example via a phone call, to be alerted about the timed out location request message.

In one embodiment, the URL link provided in the location request message can be used to confirm the location of the mobile communication device, even after the duration of the defined time period. In one embodiment, after a timeout outcome, the server 102 sends another location request message to the mobile communication device to re-alert the POI 108. Server 102 maintains a re-try limit, indicating the number of times that the server 102 will re-send the SMS message before completing process 700. The server will stop re-sending SMS messages once that re-try limit is reached.

A ‘no geographic information’ outcome 704 occurs when the server 102 does not receive geo-location coordinates from the mobile communication device 108 within a defined time period. This can occur if the operator of the mobile communication device 108 does not allow access to the GPS sensor within the time period.

Depending on the configuration settings associated with the POI 108, a ‘no geographic information’ outcome may not necessarily be a breach of the monitoring conditions it will be recorded as a Low-Level Alert (LAL) and will be escalated to DMA. The device owner can be contacted directly to receive further instructions on how to provide permission for the location determination webpage code to access the GPS sensor of the mobile communication device 108.

In step 705, the server 102 receives 118 the geo-location coordinates from the mobile device 102. In step 707, the server 102 determines whether the received geo-location coordinates are within the acceptable location boundary associated with the POI 110.

A ‘location outside acceptable boundary’ outcome 706 occurs when the server 102 determines, in step 707, that the geo-location coordinates, provided by the mobile communication device within the location confirmation reply message 118, indicate a location that is outside the acceptable location boundary associated with the POI.

As this event is a breach of the monitoring conditions, it will be recorded as a High-Level Alert (HLA). It will be escalated to DMA who will seek to contact the device owner directly. It will also be escalated to EA which will follow the appropriate procedure for the breach violation.

A success outcome 708 occurs when the server 102 determines that the geo-location coordinates, provided by the mobile communication device within the location confirmation reply message 118, indicate a location that is within the acceptable location boundary associated with the POI. This outcome will be recorded in the location confirmation log.

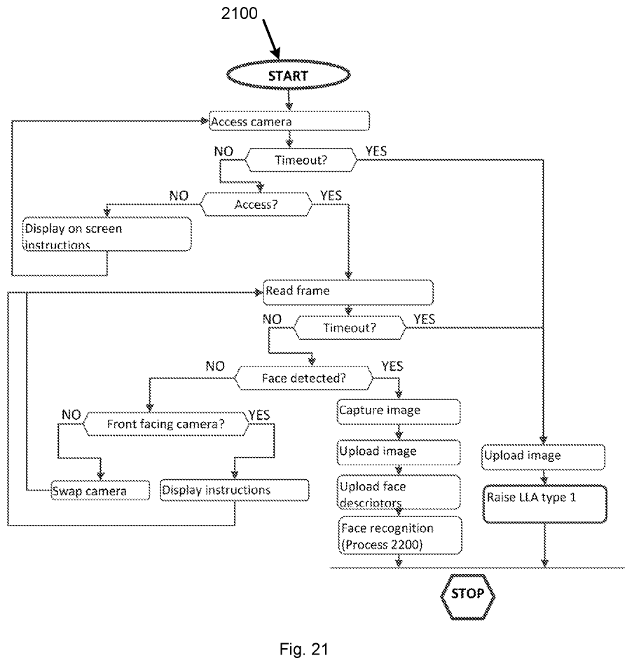

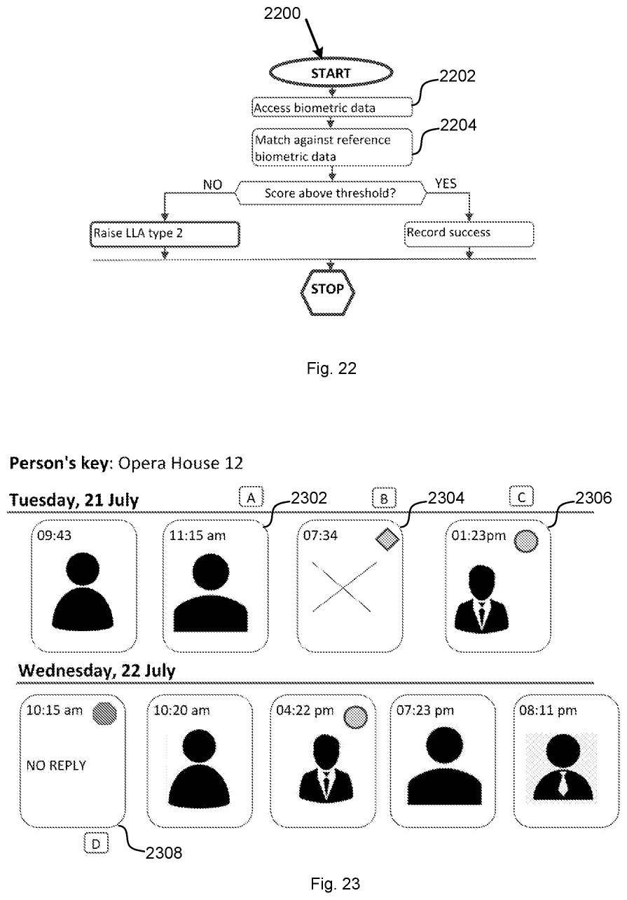

Depending on the configuration settings for the POI, the location confirmation process may collect and compare biometric data, in step 710, before considering that the location confirmation process has successfully completed. The biometric data collection process 2100 is described in relation to FIG. 21. The biometric data comparison process 2200 is described in relation to FIG. 22.

Determining Whether Location is within Acceptable Height Boundary

In step 707, the server 102 determines whether the received geo-location coordinates are within the acceptable location boundary associated with the POI 110.

In one embodiment, the acceptable location boundary is defined, at least in part, by an acceptable height range. The acceptable height range may be defined in terms of one or more barometric pressure values. When the required location for the POI is set by the DMA, the required location is associated with an initial barometric pressure measurement determined by the barometric sensor of the device 108.

In one embodiment, in step 705, the server 105 receives a barometric pressure measurement from the mobile device 108. In step 707, the server 102 compares the received barometric pressure measurement with the initial barometric pressure measurement associated with the required location of the POI. If the received barometric pressure measurement is within a POI Barometric Tolerance (PBT) of the initial barometric pressure measurement, then the server 102 determines that the mobile device 108 is within the acceptable height range of the acceptable location boundary.

In step 708, the server 102 may replace the initial barometric pressure measurement, associated with the POI, with the received barometric pressure measurement, such that a subsequently received barometric pressure measurement is compared, by the server 102 in step 707, with the previously received barometric pressure measurement to determine a change in barometric pressure.

### Barometric Neighbourhood

In one embodiment, the DMA determines one or more barometric neighbourhood (BN). A barometric neighbourhood is a group of POIs that are associated with required locations that are in close proximity and for which variations in barometric pressure are minimal. In one embodiment, the POIs in the barometric neighbourhood are associated with required locations that are located within a few kilometres of each other.

The DMA may further define a minimum BN radius as the smallest size for a barometric neighbourhood. Even in urban zones where high densities of POIs can be expected the server may be configured to not set barometric neighborhoods to be smaller than the minimum BN radius, as using smaller areas will not increase the accuracy of the height measurements, as indicated by barometric pressure sensors in mobile devices inside the BN.

The DMA may further define a maximum BN radius as the largest size for a barometric neighbourhood. In areas of low POI densities, such as rural areas, the server 102 will not consider areas larger than the maximum BN radius even if the number of sensors in the BN is not very large, as any increase inaccuracy from having more barometric pressure measurements from mobile devices within the BN will be lost due to the fact that the chance of having natural variation in pressure between points in the BN will increase.

The DMA may further define a discrete set of points, t, in time for the computation of various BN parameters such as BND and collecting temperature information.

The POI Barometric Tolerance (PBT) may be defined by the DMA as the largest acceptable variation between the initial barometric pressure measurement and the received barometric pressure measurement relative to the Barometric Neighbourhood that the POI is associated with. The PBT may be expressed in pressure units or in meters. From the perspective of monitoring self-isolation of a POI, the PBT may be measured in meters, with an example PBT equaling+/−3 meters.

The conversion of the PBT expressed in pressure units and its equivalent height tolerance needs to consider the ambient temperature around the mobile device 108. In one embodiment, the geo-location code, and the server 102 assumes that the ambient temperature around the mobile device 108 is 20-22 degrees Celsius. In this way the measurement of the temperature via an additional sensor is not required.

If the server 102 determines, in step 707, that the mobile device 108 is located at a height that is outside the PBT for the POI, then the server 102 may raise an alert by progressing to the ‘location outside acceptable boundary’ outcome in step 706.

In one embodiment, the server 102, in step 707 determines the height of the mobile device 108, in accordance with the following steps. The height of the mobile device 108 may be indicative of the location of the POI within a multi-storied building.

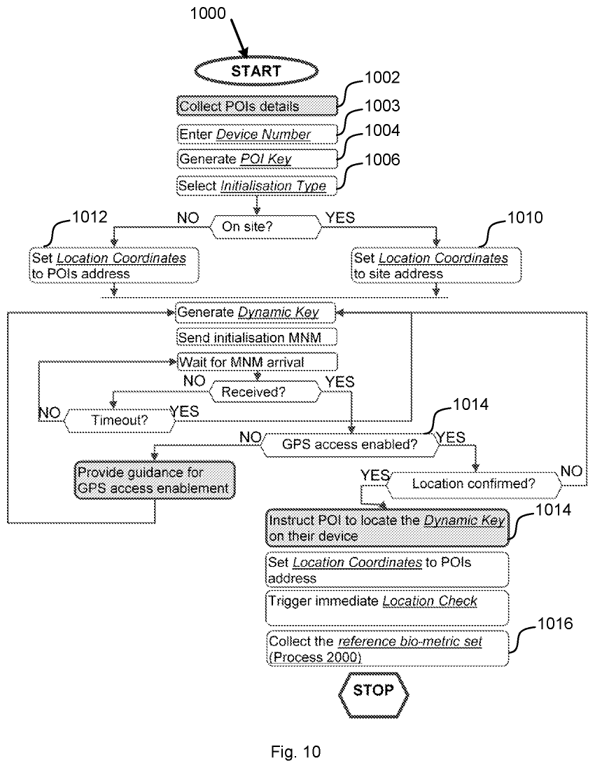

In one embodiment, during the process of registering a POI with the location confirmation process and initialising a location confirmation message sequence (as described below in relation to FIG. 10), the server 102 sends the ‘first location confirmation MNM/SMS’ message. The mobile device 108 replies to this message with the GPS coordinates of the mobile device, as determined by the mobile device's GPS sensor, and an initial barometric measurement, as determined by the mobile device's barometric sensor.

The server 102 associates the POI to one or more Barometric Neighbourhoods. In one embodiment, the server 102 periodically computes the Barometric Neighbourhoods Delta as the average of the difference between POI's pressure (PP) readings at consecutive points in time (T−1) and T, in accordance with the following formula, which applies the arithmetic average, e.g. arithmetic mean, as a possible implementation of the algorithm only.

$$BND ( t ) = {\sum }_{1}^{N} ( PP ( t ) - PP ( t - 1 ) ) / N$$

wherein N is the number of sensors (or POIs) in the BN, and t represents a discrete set of points in time for the computation of BND. This set can be used system wide.

On receiving data from a location check, the server 102, computes the POI Delta as the difference between pressure (P) readings at consecutive points in time (t−1) and t, in accordance with the following formula:

PD(t₂)=PP(t₂)−PP(t₂−1)
wherein t₂ represents the discrete set of points in time for a POIs location check, t is system or BN wide and t₂ is specific to a POI. There may be no correlation between t and t₂.

The server 102 further computes the average variation between PD and the latest BND from all BNs to which the POI is associated, to determine the Relative POI Delta (RPD). The latest BND(t_{M}) may be defined as the BND with t_{M}=max(t) where t<=t₂. The server 102 may determine the RPD in accordance with the following equation:

$$RPD ( {t}_{2} ) = {{\sum }_{1}}^{Q} ( \vert PD ( {t}_{2} ) - BND ( {t}_{M} ) \vert ) / Q$$

wherein Q is the number of BN to which the POI belongs.

If the server 102 determines that RPD(t₂) larger than BPT, then the server 102 may raise a high level alert (HLA).

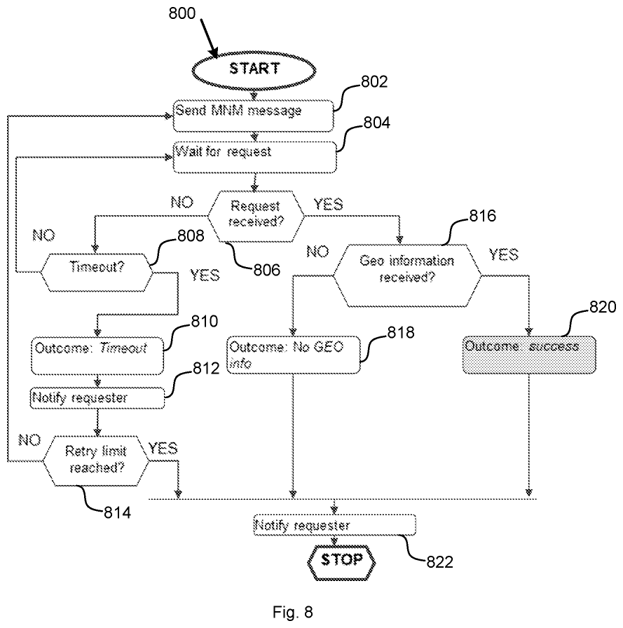

### FIG. 8—Location Confirmation for Providing Assistance

In accordance with another embodiment of the disclosure, the system 100 is used to determine the location of a POI 110 for the purpose of assisting the POI. This functionality may find application in search and rescue operations, in which the location of a POI is required so that the POI can be assisted or rescued.

FIG. 8 is a flowchart illustrating the process 800, as performed by the server 102, for a location search of a POI 108, according to an embodiment. The server 102 executes process 800 iteratively, such that a further iteration of process 800 may be performed upon completion of an iteration of loop 800.

In step 802, the server 102 sends a message to the NM provider 104, which formats the message as an SMS, and sends the SMS message to the mobile communication device 108. In step 804, the server waits for a HTTP request from the mobile communication device 108, which indicates that a user of the MCD has activated the URL link provided in the SMS message send in step 802. If the server 102 receives a HTTP request from the mobile communication device 108, the server 102 sends the webpage, including the geo-location code, to the mobile communication device 108. In step 806, the server 102 waits for a reply from the mobile communication device 108.

If a reply is received, the server determines, in step 816, whether the reply includes geo-location data. If the reply includes geo-location data, the outcome 820 of the process 800 is successful, meaning that the location of the POI has been confirmed for the purposes of providing assistance to the POI. The server may record the geo-location data in a log, and notify the requester in step 822.

If the operator of mobile communication device 108 does not activate the URL link in the allocated time, a timeout outcome 808 occurs. As noted above, in relation to process 700, while the existing MNM/SMS can still be used to confirm the location, a new message can be sent so that the device can re-alert the POI.

Outcome 818 occurs when the mobile communication device 108 fails to send geo-location data to the server 102. This may occur if the operator of the mobile communication device 108 does not allow access to the GPS sensor in a timely fashion.

In the context of search and rescue operations, is may be likely that the POI is willing to cooperate to identify the POI's location. Accordingly, the use of an enforcement authority to prevent fraud is not applied in this scenario. In this case only the decision making authority (DMA), which may be a rescue team member, is involved. As a result, process 800 is simpler than process 700.

### Usage of Previously Sent MNM Messages

A previously sent or activated MNM message, that is present on the mobile communication device 108, can be used by the operator of the device 108 to re-trigger a location check. This feature is used, as described below for re-establishing channel trust.

In such cases, the location of the mobile communication device will be determined by the mobile communication device 108 executing the geo-location code and transmitting the geo-location data to the server 102, via the methods described above. However, when the server 102 receives a reply triggered from an expired MNM message, the server will record the reply in the log as being a reply that was initiated by the operator of the mobile communication device 108 rather than the location confirmation system.

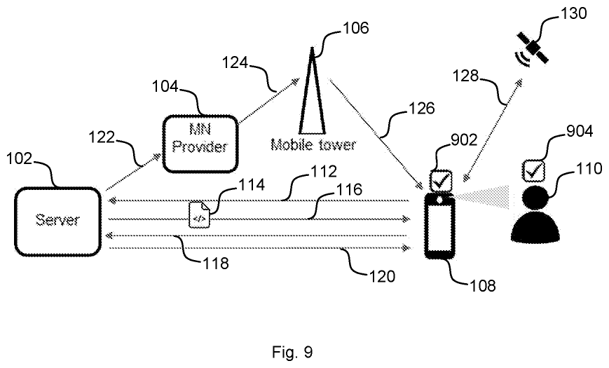

### FIG. 9—Privacy Protection Points

FIG. 9 illustrates privacy protection points for location confirmation system 100, according to an embodiment. At privacy checkpoint 1, indicated by reference numeral 902, the location confirmation request is started by the mobile communication device 108 receiving an SMS message 126. This signals that a location confirmation process has been initiated by the server 102. At this stage, there is no geo-location code, or other code relating to the location confirmation check, code running on the device 108.

At privacy checkpoint 2, indicated by reference numeral 904, the location confirmation check process is triggered by the operator of the device 108 activating the URL link (e.g. by tapping on the link) provided in the received SMS message 126.

In response to the operator activating the link, the geo-location code will be downloaded from the server 102 and executed on the mobile communication device 108. Advantageously, these processes will only be triggered after action has been taken by the operator of the device. Accordingly, the operator of the device has control over whether the location confirmation process occurs.

### Spoofing Prevention

Spoofing is the act of disguising a communication from an unknown source as being from a known, and trusted source. In the context of the present disclosure, an example of a spoofing attack is when a party, other than the server 102 (i.e. the spoofing party), sends a MNM message to the device 108, wherein the MNM message may appear to be a location confirmation message from server 102.

The POI receiving the spoofing MNM message may activate a link included within the message. The link may download or activate malicious code. The malicious code may collect information from the device 108 and provide such information to the spoofing party. For example, the activated code may relay the position of the device to the spoofing party. Such spoofing activity is undesirable.

The location confirmation system may be used by government or legal agencies to monitor the location of POIs. Such agencies have a strong authoritative role, and spoofing messages that purport to come from these agencies may be readily acted on by the POI. Accordingly, it is desirable that protections exist within the location confirmation system, so that the POI can readily distinguish spoofing messages from genuine messages.

### Protecting the Sequence of Messages

In one embodiment, the location confirmation system provides anti-spoofing provisions for a sequence of location confirmation messages, so that the POI can identify if a spoofing message has been inserted into a sequence of legitimate messages from server 102.

The server 102 uses matching sets of keys included in sequential messages, such that a message includes a key, and a subsequent message in the message sequence includes that same key. In this way, a location confirmation message delivers an authentication mechanism across a sequence of location confirmation messages, so that spoofed messages cannot be inserted in the message sequence.

The key is changed for each message, so that spoofing messages cannot be inserted within the sequence. The keys may be dynamically generated by the server 102, or may be a key from a pre-generated list of keys. The message sequence keys can be abstract or natural language keys. Natural language keys provide familiarity and ease of readability for users. Abstract keys may be more appropriate for deployment in applications that require large scale involvement of the population with different linguistic backgrounds.

### Trusted Source

Another anti-spoofing mechanism provides the message receiver with confidence that the sequence of messages originated from a trusted source. In one embodiment, the server 102 includes a POI key within the location confirmation message. They POI key is an identifier which has been uniquely associated with the POI by the server 102 during registration of the POI to the location confirmation system. The POI key may be a name, an identification number, or an identification code.

A fixed POI key, which, for simplicity, can be a common language word. In some embodiments it is not recommended to use the POIs name instead of a random word since there is a quite probability that an attacker can acquire databases of names and personal mobile numbers. The availability of this kind of information will make the usage of the person name a much less secure alternative. In addition, by not containing names, data managed in the system may be less attractive to other types of cyber-attacks.

### POI Key and Dynamic Key

In one embodiment, the server 102 uses both a POI key, a message sequence key, and an authentication keyword (also known as a dynamic key) when sending location confirmation messages to a POI. The POI key and the dynamic key(s) can be abstract sequences of numbers and/or letters or consist of one of more spoken language words or colloquial expressions in a language familiar for the POI.

### Start-Up Kit

In the following sections, the processes for initialising the sequence of location confirmation messages, authenticating a message and re-establishing channel trust are described. In one embodiment, the process for initialising a sequence of location confirmation messages occurs when the DMA determines that a person is a person of interest for the purposes of location monitoring.

The information used by the server 102 to initialise a sequence of location confirmation messages includes, the start-up kit (described below), an identification number of the device 108 (for example, the mobile phone number of a SIM installed in the device 108), and a dynamic key included in the initial message and valid for the authentication of the next message in a message sequence.

The set of information that may be used by the server 102 to establish the location confirmation process is called the ‘start-up kit’. In one embodiment, the start-up kit includes a fixed POI key, a language preference of the POI, and contact information of the DMA to allow the POI to contact the DMA. The start-up kit further comprises a first location confirmation message to start the location confirmation message sequence. The start-up kit is supplied to the server 102 by the DMA, and is considered, by the server 102, to be trusted information.

### Message Sequence Initialisation Type

Preferably, the first location confirmation message is sent to the device 108 when the device 108 and the POI is in a trusted setting. A trusted setting is a setting in which the POI's identity can be verified to the satisfaction of the DMA, and the identification of a mobile communication device 108 associated with the POI can be established. In some situations, a trusted setting is also a setting in which the POI's location can be established, at the time of initialising the message sequence.

A trusted setting can be established, and a location confirmation message sequence can be initialised, via various initialisation types including an on-site initialisation or a remote initialisation.

In an on-site initialisation of a location confirmation message sequence, the trusted setting is established when the POI is physically present at a registration location managed by the DMA. In a remote initialisation, the trusted setting for message sequence initialisation is established when the POI is not physically present at a registration location managed by the DMA.

In one example of an on-site initialisation, a trusted setting is established when a POI is in the physical presence of DMA representative. The DMA representative may be a person, written or digital information, or software available, at a known location, to guide the POI through the registration process. Trusted settings may include the arrivals section of an airport, a medical site, a quarantine site or a border control site.

Sending the initialisation message when the POI is in the physical presence of a DMA representative provides confidence to both the DMA and the POI that the location confirmation message sequences are authentic and that a trusted message sequence has been established. In this case, the trust component of the message sequence initialisation is the POI's physical presence with the DMA representative at the registration location.

Sending the first message of a location confirmation message sequence when the POI is at a registration location with a DMA representative has the benefit of creating a record of the POI being present at the registration location as well as enabling any technical issues, such as access to the device GPS sensor, to be resolved during registration, for example by a DMA representative providing assistance to the POI. As noted above, the DMA representative may be a person, a digital device, written or digital information presented to the POI or any combination thereof.

In some situations, a trust setting may be a non-physical registration location which has been established as a trusted setting through other mechanisms to determine the registration location of the POI and the device 108. For example, in a remote initialisation, a registration location may be established via a remote communication session between the POI and the DMA. The remote communication session may be a phone call over a mobile phone network, a web call over the Internet, or another means to enable a remote audio and/or visual communication session. The communication session may be initiated by the POI contacting the DMA, or by the DMA initiating contact with the POI.

When the trust setting is a non-physical registration location, the trust component of the message sequence initialisation is the remove communication session between the POI and the DMA representative. In one embodiment, the DMA can establish that the POI is at a particular location by the POI receiving and responding to an initial location confirmation message while in communication with the DMA via a mobile phone call.

### FIG. 10—Message Sequence Initialisation

FIG. 10 is a flowchart illustrating a process 1000 for registering a POI with the location confirmation process and for initialising a location confirmation message sequence, according to an embodiment. Process 1000 is performed by the mobile communication device in conjunction with the server.

In one embodiment, process 1000 is performed when the POI is in a trusted location, such as an airport arrivals gate, and is performed with the guidance and oversight of a DMA device or representative. The DMA representative may be a person, written or digital information, or software available to guide the POI through the registration process.

In one embodiment, registration code is downloaded to the mobile communication device and executed by the mobile communication device, to facilitate the POI and/or DMA representative performing 1000 on the mobile communication device.

In steps 1002 and 1003, the POI or DMA representative enters the POI's details and the identification number of the mobile communication device (e.g. mobile phone number). In step 1004, the registration code generates a POI key. In step 1006, the POI or DMA representative indicates whether the initialisation process is on-site or remote. In an on-site initialisation of a location confirmation message sequence, the trusted setting is established when the POI is physically present at a registration location managed by the DMA. In a remote initialisation, the trusted setting for message sequence initialisation is established when the POI is not physically at a registration location managed by the DMA.

If the initialisation is an on-site initialisation, the DMA sets the required location for the POI to be the current location of the mobile communication device. The registration code obtains the GPS coordinates of the mobile communication device and sets 1010 the required location for the POI to be the current GPS coordinates of the mobile communication device. In one embodiment, the registration code also obtains a current barometric pressure measurement from the barometric sensor of the mobile communication device 108 and sets 1010 the initial barometric pressure measurement to be the current barometric pressure measurement of the mobile communication device.

On the other hand, if the registration process 1000 is remote, i.e. performed when the POI is not physically at a registration location managed by the DMA, the POI or DMA representative indicates 1012 the required location via a map application, address field or GPS coordinates entry field. In one example, the POI's required location is the POI's home address.

Next the registration code communicates with the server 102, so that the server can perform the next steps of the registration process. The server 102 generates a dynamic key for the location confirmation message sequence, and the server 102 sends an initial location confirmation message to the mobile communication device 108.

The mobile communication device 108 receives the initial location confirmation message. In step 1014, with guidance from the DMA representative, the POIs activates the URL included in the initial location message, which obtains the GPS coordinates of the mobile communication device.

In step 1016, the registration code initiates the collection of registration biometric data of the POI, as described with regard to FIG. 20.

In some situations, it may be desirable that the registration process 1000 is performed while the POI is in contact with the DMA, so that various instructions required, such as enabling access to camera, can be provided.

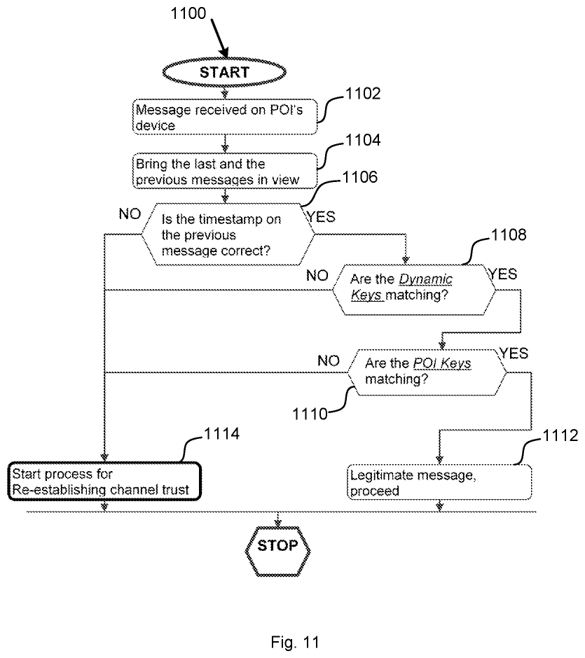

### FIG. 11—Sequence Authentication

FIG. 11 is a flowchart illustrating a process 1100, as performed by a POI operating a mobile communication device 108, for authenticating a MNM message of a sequence of location confirmation messages, according to an embodiment.

In step 1102, device 108 receives a new MNM message, which appears to originate from server 102. In step 1104, the POI, operating device 108, views the new MNM message, and the most recent previous MNM message received by the device 108. Depending upon the display capabilities of the device 108, the POI may be able to view the new MNM message, and the most recent previous MNM message simultaneously in a single view.

In step 1106, the POI performs a visual inspection of the timestamp (e.g. time of arrival) of the new message and the most recent previous message. The POI determines whether the timestamp of the previous message (roughly) matches the last time that the location of the POI was checked. Spoofing messages may have a wrong timestamp value.

Additionally, the POI may consider the timestamps of the new and the previous message to determine whether the messages have arrived in quick succession. The arrival of a new message in quick succession after the most recent previous message may be indicative of a potential spoofing attack.

If the POI identifies that the new message may be a spoofing message, the POI can take steps to re-establish trust in the communication channel, in step 1114.

In step 1108, the dynamic key included in the most recent previous message is compared to the dynamic key included in the new message. In one embodiment, the POI performs this comparison via a visual inspection. If the dynamic keys match, the POI can have confidence that the new message is part of a trusted location confirmation message sequence, and is not a spoofing message. Alternatively, if the dynamic keys do not match, the POI may have cause to consider that the new message is a spoofing message and the POI can take steps to re-establish trust in the communication channel, in step 1114.

In step 1110, the POI key included in the new message is compared to the POI key included in one or more of the previous messages in the location confirmation message sequence stored on the device 108. In one embodiment, the POI performs this comparison via visual inspection. If the POI key included in the new message matches one or more of the POI keys included in the location confirmation message sequence, the POI can then conclude, in step 1112, that the new message is part of a trusted location confirmation message sequence, and is not a spoofing message. Alternatively, if the POI keys do not match, the POI may have cause to consider that the new message is a spoofing message and the POI can take steps to re-establish trust in the communication channel, in step 1114.

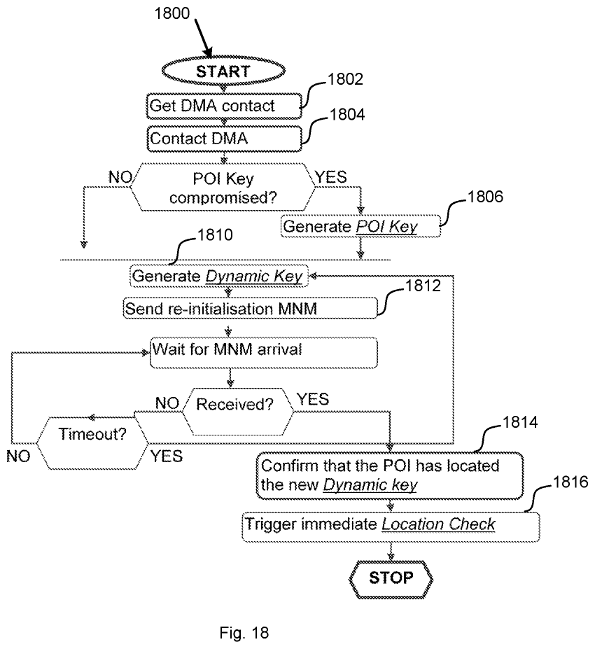

A process of re-establishing trust in the communication channel is illustrated in FIG. 18.

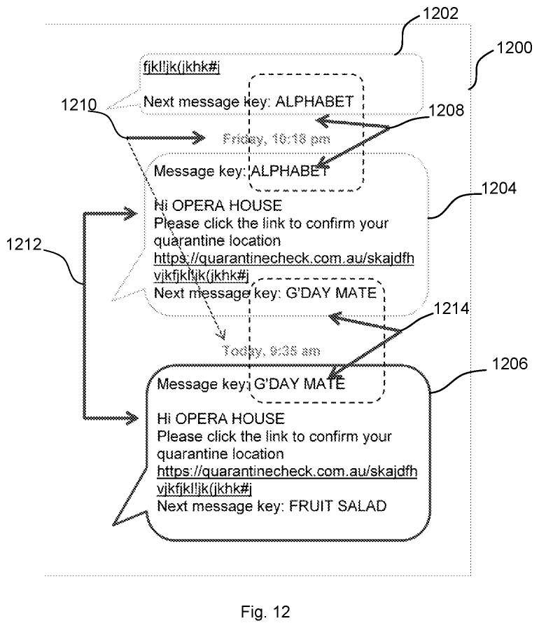

### FIG. 12—Using Words for Dynamic Keys

FIG. 12 illustrates a display, or part thereof, of mobile communication device 108, wherein the dynamic keys are words or colloquial expressions, according to an embodiment. Display 1200 displays first 1202 message, second 1204 message and third 1206 message in a sequence of location confirmation messages received on a mobile communication device 108. The dynamic key that is included in first message 1202 and repeated in second message 1204 is the word ‘ALPHABET’, as indicated by reference numeral 1208. The dynamic key that is included in second message 1204 and repeated in third message 1206 is the expression ‘G′DAY MATE’, as indicated by reference numeral 1214. The POI can visually match the dynamic keys 1208 of the first and second messages, and the dynamic keys 1214 of the second and third messages, to deduce that the first, second and third messages form a contiguous sequence of the location confirmation messages.

The POI key of the second message 1204 and the third message 1206 is ‘OPERA HOUSE’, as indicated by reference numeral 1212. The POI key of the first message 1202 is not show in FIG. 12. The POI can visually match the POI keys of the messages to deduce that the messages were sent by the server 102.

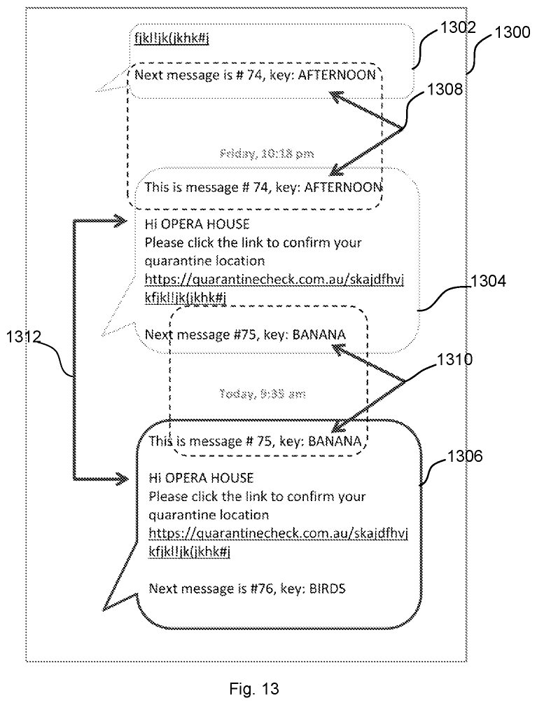

### FIG. 13—Using Sequence Numbers and Words for Dynamic Keys

FIG. 13 illustrates a display, or part thereof, of mobile communication device 108, wherein the dynamic keys are words and sequence numbers, according to an embodiment.

Display 1300 displays first 1302 message, second 1304 message and third 1306 message in a sequence of location confirmation messages received on a mobile communication device 108. The dynamic key that is included in first message 1302 and repeated in second message 1304 is the word ‘AFTERNOON’ and a sequence number ‘#74’, as indicated by reference numeral 1308.

Also included in second message 1304 is the dynamic key for the next message (third message 1306) in the sequence of location confirmation messages. This dynamic key is indicated by reference numeral 1310 and comprises a word, ‘BANANA’ and a sequence number. Notably, the sequence number of the dynamic key shared by second message 1304 and third message 1306 is incremented with reference to the sequence number of the dynamic key shared by first message 1302 and second message 1304.

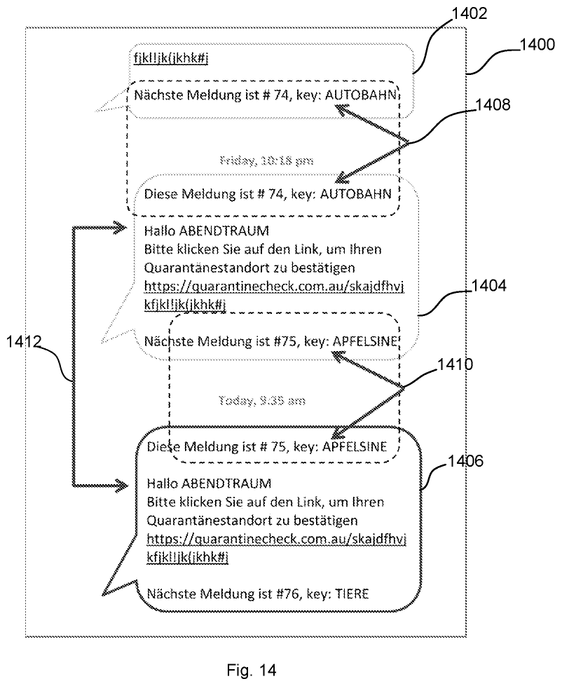

### FIG. 14—Using a Preferred Language

FIG. 14 illustrates a display, or part thereof, of mobile communication device 108, wherein the dynamic keys are provided in a language preferred by the POI, according to an embodiment. The dynamic keys included in messages 1402, 1404 and 1406 are words provided in German.

As noted above, the start-up kit includes information which indicates the preferences of the POI, including a language preference. In one embodiment, the dynamic keys are words of the POI's preferred language, as indicated by the POI's language preference. Advantageously, POIs from a non-English speaking background can be accommodated by the location confirmation system.

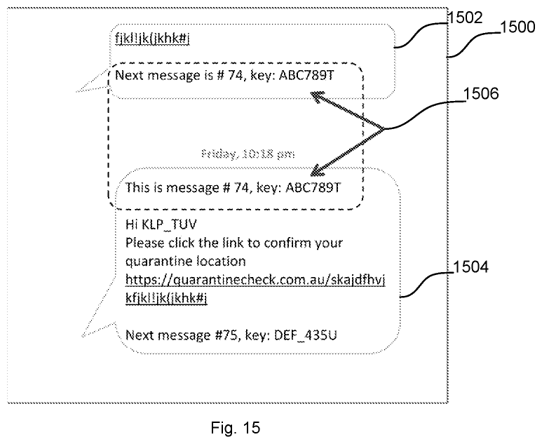

### FIG. 15—Using Abstract Dynamic Keys

FIG. 15 illustrates a display, or part thereof, of mobile communication device 108, wherein the dynamic keys are an abstract sequence of letters or numbers rather than words, according to an embodiment. Display 1500 displays first message 1502 and second message 1504 in a sequence of location confirmation messages received on a mobile communication device. The dynamic key that is included in first message 1502 and repeated in second message 1504 is the abstract sequence of letters and numbers ‘ABC789T’ and a sequence number ‘#74’, as indicated by reference numeral 1506.

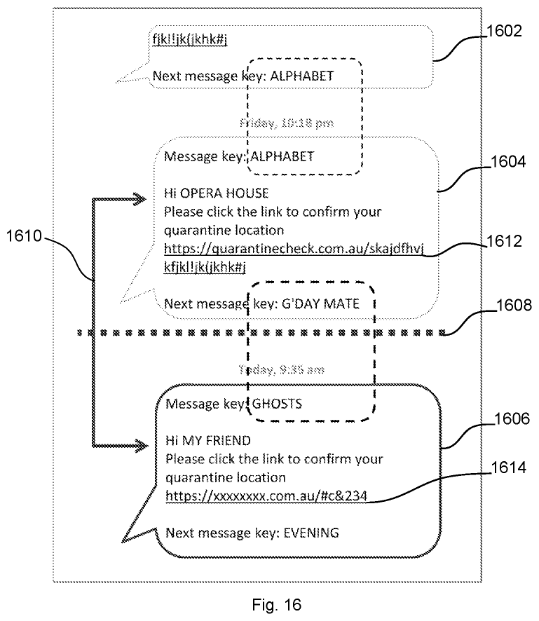

### FIG. 16—Break-In Point

A break-in point is the boundary between the last legitimate location confirmation message received by a mobile communication device from the server 102 and a spoofing message received by a mobile communication device. Using the techniques described herein, a break-in point may be detected by a visual inspection of the received messages by the POI, or other operator of the mobile communication device 108.

FIG. 16 illustrates a display, or part thereof, of mobile communication device 108, wherein the break-in point may be identified by the POI 110 via one of three visual clues, according to an embodiment. Display 1600 displays first 1602 message, second 1604 message and third 1606 message in a sequence of location confirmation messages received on a mobile communication device 108. First message 1602 and second message 1604 are legitimate messages, sent by server 102. In contrast, third message 1606 is a spoof message, send by an unknown party. Accordingly, the break-in point 1608 is between second message 1604 and third message 1606.

A visual clue indicating that the third message 1606 is a spoof message is that the next dynamic key ‘G'DAY MATE’ included in second message 1604 differs from the dynamic key ‘GHOSTS’ included in third message 1606. A further visual clue indicating that the third message 1608 is a spoof message is that the POI key ‘OPERA HOUSE’ included in the second message 1604 differs from the POI key ‘MY FRIEND’ included in the third message 1606.

A third visual clue indicating that the third message 1608 is a spoof message is that the URL 1614 provided in the third message 1606 differs from the URL 1612 provided in the second message 1604.

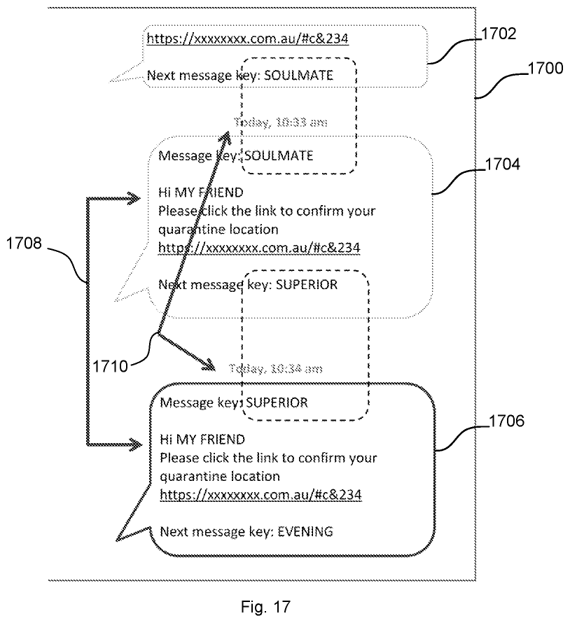

### FIG. 17—Flood of Spoof Messages

A spoofing party can attempt to flood the mobile communication device 108 with multiple location confirmation messages. A multitude of spoof messages may push the break-in point outside the portion of the sequence of messages that is readily displayed on the mobile communication device 108 for viewing by the POI 110. As a result, the POI 110 will be presented with what may appear to be a legitimate sequence.

FIG. 17 illustrates a display, or part thereof, of mobile communication device 108, showing three spoof messages 1702, 1704, and 1706, according to an embodiment. The dynamic keys of sequential messages shown within the bounds of display 1700 match. Additionally, the POI key of the messages shown within the bounds of display 1700 match. Furthermore, the URLs of the shown messages 1702, 1704, and 1706 match. Accordingly, based on these visual clues, in the context of the shown messages 1702, 1704, and 1706, the POI 110 may deduce that messages 1702, 1704, and 1706 are all legitimate location confirmation messages sent by server 102. However, the POI 110 may notice that the timestamps of the messages are very close to each other. In order to avoid detection, the spoofing party may send multiple messages in quick sequence. As a result, timestamps will have very narrow time gaps.

The POI 110 may notice that the POI key provided in the messages is not the POI provided by the DMA during registration. The POI key is the authentication element that the spoofer cannot forge even in the fake message sequence scenario described above. Despite its key role it still can be stored by POIs even in printed form to remove the need to memorise it. Assuming that the POI key is stored in printed the only way of a spoofer to get in possessing of this information is by physical access. Even if the spoofer knew the POI key, the spoofer would still need to know the device/phone number of the POI to launch a successful spoof attack.

### FIG. 18—Re-Establishing Channel Trust

FIG. 18 is a flowchart illustrating a process 1800, as performed by server 102 in conjunction with a mobile communication device 108, for re-establishing channel trust between the server 102 and the mobile communication device 108, according to an embodiment.

The process 1800 may be performed in scenarios in which either the channel trust was affected as a result of cyber-attack attempts or any other cases in which a POI will request it. Once a spoofing message has been identified by the POI, the POI can request to perform process 1800 to re-establish the channel's trust.

In steps 1802 and 1804, the POI determines the DMA contact details and contacts the DMA using the details presented on the start-up kit. Alternatively, the DMA contact details may be obtained online.

If the POI reports to the DMA that the POI key has been compromised, in step 1806, the DMA generates a new POI key. The DMA uses the location confirmation process to generate a new dynamic key, in step 1810, and sends a new initialisation location confirmation message 1812 to the mobile communication device 108. Once the mobile communication device 108 receives 1814 the initialisation message, the POI activates the URL included in the initialisation message to trigger a location check 1816 and complete the location confirmation process.

### Requesting Initialisation

There are situations in which it is desirable for the DMA to initiate contact with potential persons of interest. An example of a such a situation is when a pandemic or epidemic is present in a population, and the DMA is tasked with identifying persons who may have come into contact with an infected or potentially infected person, so as to mitigate the spread and impact of the infectious disease.

In such situations, the DMA may send a ‘request to initialise’ message to a person who the DMA considers to be a potential person of interest. The message requests that the POI initiate a communication session with the DMA. In response to the POI making contact with the DMA, the DMA can perform remote registration of the POI and can, if necessary, perform remote initialisation for a sequence of location confirmation messages. Accordingly, the DMA can remotely establish a trusted setting in which to initialise a message sequence.

It is noted that often a person is reluctant to respond to messages that were sent by an unknown or unexpected sources. Such unsolicited messages are often viewed as unwelcome cold-calls, robo-calls, spam or phishing messages. Accordingly, it is desirable to distinguish a ‘request to initialise’ message from the DMA from other unsolicited messages that a person may receive.

In one embodiment, the server, acting on behalf of the DMA, includes shared-knowledge information in the ‘request to initialise’ message to afford a level of legitimacy to a message, in the eyes of the POI. The shared-knowledge information may be information that is known to the POI, but the POI would not consider the information to be widely known. In some embodiments, the shared-knowledge information may be a secret that is known only by the POI and the DMA. The shared-knowledge information suggests, to the POI, that the message has been sent specifically for the POI by a sender that has specific knowledge of the POI. Accordingly, the POI may have an reduced inclination to dismiss or ignore the ‘request to initialise’ message from the DMA.

The ‘request to initialise’ message also includes information which requests that the recipient contact the DMA, and may also provide contact information indicating how the recipient can make contact with the DMA.

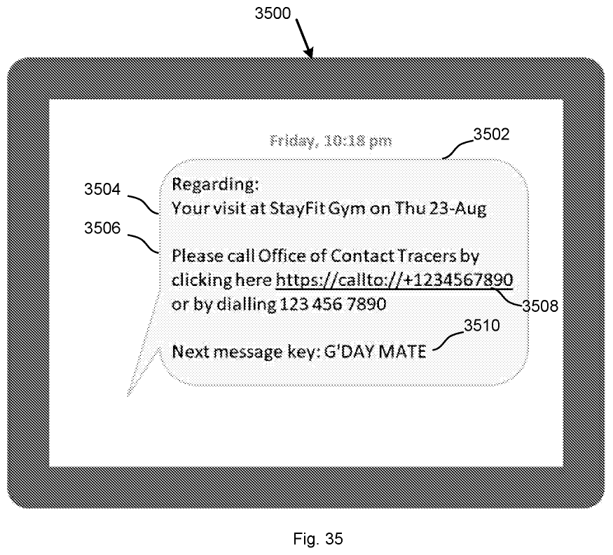

FIG. 35 illustrates a display 3500, or part thereof, of mobile communication device associated with a POI, on which a ‘request to initialise’ message 3502 is received from the DMA, according to an embodiment. In the embodiment illustrated in FIG. 35, the POI is not yet registered as a POI by the DMA for the purposes of receiving location confirmation messages. The DMA is seeking to remotely initialise a sequence of messages with the POI.

The ‘request to initialise’ message 3502 includes shared-knowledge information that is known to both the DMA and the POI. In the example shown in FIG. 35, the shared-knowledge comprises details of a venue visit by the POI. Specifically, the shared-knowledge 3504 is that the POI visited the StayFit Gym on Thursday 23 August. The DMA may know this information due to having access to attendance logs at this location, or the DMA may have become aware of this information through other means.

In another example, the shared-knowledge information may comprise: the name of a known contact of the POI; details of the POI's place of employment; details of a membership, such a membership of a social club; or details relating to the POI's dependent, such as the name of the dependent's school.

The message 3502 further comprises a request 3506 to make contact with the DMA. The POI can establish contact with the DMA directly from an embedded link 3508 that contains the correct number or alternatively, the actual phone number can also be displayed to allow manual dialling. In this way, the ‘request to initialise’ message 3502 encourages the POI to make contact with the DMA by providing legitimacy to the message.

In response to the POI making contact with the DMA, the DMA can perform the remote registration of the POI and can, if necessary, perform remote initialisation of a message sequence such as a location confirmation message sequence.

The message 3502 may further comprise a dynamic key 3510 which can be repeated in the next message sent from the DMA to the POI to provide the POI with assurances that the subsequent message originated from the DMA. Dynamic keys are described in relation to FIGS. 12 to 15.

### Fraud Prevention

The present disclosure provides a system and method for identifying the location of a POI by determining the location of a mobile communication device associated with the POI. However, to correctly determine the location of the POI, in some situations, it may also be necessary to determine that the operator of the mobile communication device is the POI, rather than some other person. If the location confirmation step is performed by a person other than the POI, the location confirmation would be a false confirmation.

In one embodiment of the system, the location confirmation process further includes the steps of the server 102 obtaining a biometric attribute of the operator of the mobile communication device, and comparing the biometric attribute of the operator to a reference biometric attribute associated with the POI 110. If the biometric attribute of the operator matches the reference biometric attribute associated with the POI 110, the server 102 can establish the authenticity of the person confirming the location check.

In some situations it may be undesirable to obtain and retain biometric attributes of a POI, for example, due to privacy concerns. Accordingly, in some embodiments, the server 102 does not match the biometric attributes to a real person's identity. Instead, it will compare those attributes with a reference set that is collected at the registration time. In other words, it will verify that the person confirming the location is the person that was present at registration with DMA without needing to know who the person is.

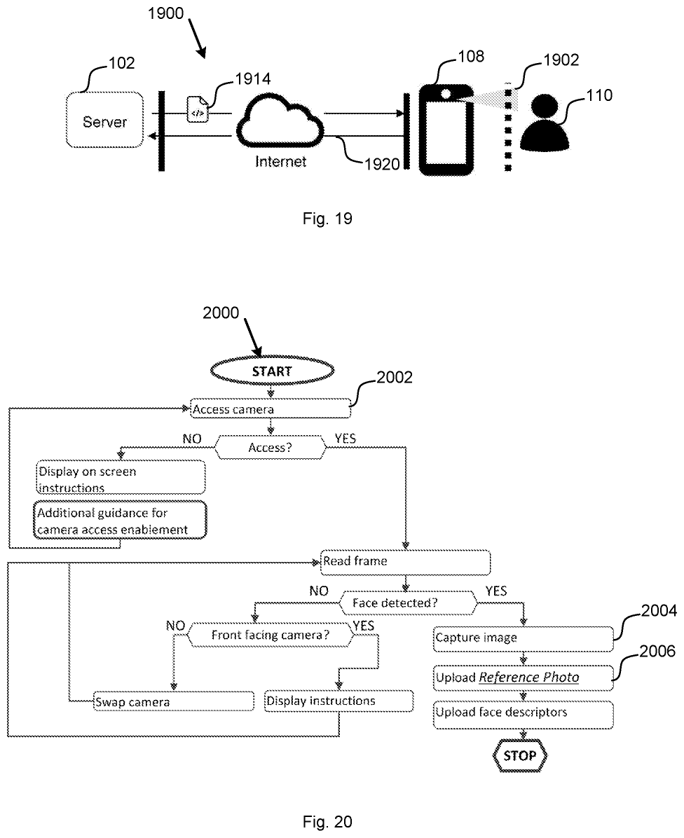

### FIG. 19—Biometric Data Collection

FIG. 19 is a system diagram illustrating the components of a system 1900 in which the location confirmation process includes the steps of obtaining and verifying biometric data, according to an embodiment. The webpage 1914 downloaded to the mobile communication device 108 from the server 102 comprises biometric collection code. After executing the geo-location code, the mobile communication device executes the biometric collection code. The biometric collection code accesses at least one of the sensors of the mobile communication device 108 to obtain biometric data of the operator. The sensors may include a camera, fingerprint scanner, microphone.

In the embodiment illustrated in FIG. 19, the mobile communication device 108 uses the camera to obtain 1902 a photo of the face of the operator 110. In one embodiment, the mobile communication device 108 performs facial detection processing on the photo to determine whether the operator's face has been photographed suitably.

The mobile communication device 108 sends 1920 the GPS co-ordinates and the face photo to the server 102. The server 102 confirms that the photographed face matches the facial recognition markers for the POI, as stored on the server 102.

### FIG. 20—Collecting the Reference Biometric Data

FIG. 20 is a flowchart illustrating a process 2000, as performed by a POI operating a mobile communication device 108, for collecting a reference set of biometric data to be associated with the POI, according to an embodiment. In this embodiment, the biometric data is facial recognition markers.

Process 2000 is performed during registration of the POI to the location confirmation process. Registration to the location confirmation process may occur in a trusted location, such as an airport arrivals gate, and may be performed with the guidance and oversight of a DMA device or representative.

In one embodiment, during registration of the POI to the location confirmation process, registration code is downloaded from the server 102 to the mobile communication device 108. The registration code performs process 2000. In step 2002, the registration code accesses the camera of the mobile communication device and captures an image of the POI's face. The registration code uploads 2006 a reference photo to the server 102. If the registration code cannot access the devices camera, the registration code provides on-screen instructions to the POI on how to enable camera access.

### FIG. 21—Collecting Biometric Data

FIG. 21 is a flowchart illustrating a process 2100, as performed by a POI operating a mobile communication device 108, for collecting biometric data from the operator of the mobile communication device 108, according to an embodiment.

Process 2100 has a timeout. In case of a timeout process 2100 will complete without a face being detected. The system will receive the current camera frame, if any, and the location confirmation process 700 will complete with a successful location confirmation; however the process 700 will raise a Low-Level Alert (LLA) indicating that the biometric data was unable to be confirmed.

### FIG. 22—Comparing Biometric Data

FIG. 22 is a flowchart illustrating a process 2200, as performed by server 102, for comparing biometric data received from the mobile communication device 108 with reference biometric data stored on server 102, according to an embodiment. In step 2202, the server 102 accesses the biometric data from the POI's mobile communication device. In step 2204, the server 102 compares the accessed biometric data to the reference biometric data associated with the POI.

During the comparison process 2200, if the matching threshold is below a predefined matching threshold, a Low-Level Alert type 2—Person not recognised, will be issued by server 102. Server 102 does not store any personal details. For matching, the server 102 uses the minimalist biometric data collected in the reference set and no other external information. Additionally, server 102 uses only the generated POI Key for internal identification purposes.

### Multiple Biometric Attribute Types

In some situations it may be desirable to obtain and retain more than one biometric attribute of a POI, where the biometric attributes corresponding to different biometric marker types. The retention of more than one biometric attribute of a POI as reference biometric attributes may allow the server 102 to optionally compare more than one biometric attribute and compare each collected biometric attribute to the corresponding reference biometric attributes stored on the server 102.

The multiple biometric attributes may be of related types, for example a fingerprint of the left pointer finger and a fingerprint of the right pointer finger. Alternatively, or additionally, the server may retain reference markers, associated with a POI, for biometric attributes of different types, for example a fingerprint and a voice profile.

Example biometric marker types include: fingerprints of one or more different fingers, where each fingerprint is a different biometric marker type; one or more iris scans, where each iris scan is a different biometric marker type; facial recognition markers; voice profile markers; one or more retina scans, where each retina scan is a different biometric data type; signature recognition markers; hand geometry markers; ear recognition markers, where each ear recognition marker is a different biometric data type; DNA markers; gait markers; and any combination of these biometric attributes which can be used as a combined biometric marker.

In one embodiment, in step 2202, the server 102 selects a first biometric attribute type for which biometric data is to be collected from the POI. The first biometric attribute type selected by the server 102 is one of the plurality of biometric attribute types for which the server retains reference biometric data associated with the POI. The server 102 collects biometric data from the POI in accordance with the first biometric attribute type. The server 102 may then proceed with a comparison of the collected biometric data with the reference biometric data, in accordance with comparison techniques associated with the first biometric attribute type.

In one embodiment, in a subsequent execution of step 2202, the server 102 may select a second biometric attribute type for which biometric data is to be collected from the POI. The second biometric attribute type selected by the server 102 is one of the plurality of biometric attribute types for which the server retains reference biometric data associated with the POI. Additionally, the second biometric attribute type is different to the first biometric attribute type. The server 102 collects biometric data from the POI in accordance with the second biometric attribute type. The server 102 may then proceed with a comparison of the collected biometric data with the reference biometric data, in accordance with comparison techniques associated with the second biometric attribute type.

For example, in one location confirmation process, the server 102 may request that the operator of the mobile communication device verify their identity via a fingerprint. In a subsequent location confirmation process, the server 102 may request that the operator of the mobile communication device verify their identify via facial recognition.

In another embodiment, in step 2202, the server 102 selects a plurality of biometric attribute types for which biometric data is to be collected from the POI. The plurality biometric attribute types selected by the server 102 may be a subset of the plurality of biometric attribute types for which the server retains reference biometric data associated with the POI. The server 102 collects biometric data from the POI in accordance with the plurality biometric attribute types.

For example, in one location confirmation process, the server 102 may request that the operator of the mobile communication device verify their identity via facial recognition and via a voice profile.

Collecting and comparing biometric data associated with more than one biometric attribute type may enhance the verification that the person operating the mobile communication device 108 is the POI associated with the reference biometric data. Additionally, altering the type, or types, of biometric attributes that are compared during the verification process for subsequent location confirmation messages may provide additional security in the event that verification via one biometric attribute becomes insecure or can be circumvented by the operator of the mobile communication device.

### FIG. 23—Visual Location Confirmation Log

FIG. 23 illustrates a component of the user interface of the location confirmation process, executing on server 102, which depicts a visual location confirmation log for a POI, according to an embodiment.

The results of nine location confirmation checks for a POI are visually depicted, wherein each curved square shows the results of a single location confirmation check. Location confirmation check 2302 was successfully completed, with the POI being within the acceptable location boundary, and the biometric data obtained from the mobile communication device 108 matched the reference biometric data stored for the POI.

Location confirmation check 2304 was not successfully completed. Biometric data was not provided to the server 102 from the mobile communication device 108. Accordingly, a Low Level Alert type 1 was issued in relation to this location confirmation check.

Location confirmation check 2306 was not successfully completed because the biometric data sent from the mobile communication device 108 did not match the reference biometric data stored by the server 102. Accordingly, a Low Level Alert type 2 was issued in relation to this location confirmation check.

Location confirmation check 2308 was not successfully completed as no reply was received from the mobile communication device 108. Accordingly, a Low Level Alert type 3 was issued in relation to this location confirmation check.

### Boundary Crossing Management

The sections of the disclosure, provided above, describe a method and system for determining the location of a POI. The following sections of the present disclosure provide systems and methods for managing boundary crossings. In one embodiment, the boundary crossing management method is performed by server 102 in its role as a boundary crossing management server.

More specifically, the present disclosure provides a method and system for facilitating quick boundary crossing by unrestricted persons, whilst inhibiting boundary crossing by a restricted person.

### Restricted Persons

In some situations, a person of interest may be placed under certain restrictions that require a person to remain within a permitted region, or restrict the movement of a person into one or more restricted regions. Such a person is considered to be a restricted person with regard to the one or more regions that the person is restricted from entering.

In one example, a POI is subject to a quarantine order due to medical reasons. The POI is subject to a movement restriction such that the POI must remain within a defined permitted region, which may be defined as an acceptable radius around a required location. The defined permitted region may further be defined by a height value or range, relative to the surface of the earth, or a barometric pressure value or range, which is indicative of a height value or range.

Furthermore, the POI may be subject to restrictions, such that the POI is restricted from crossing a state border. Accordingly, with respect to the state border, the POI is considered to be a restricted person.

In one example, the POI may be restricted from crossing a state border, but is permitted to cross a city border. Accordingly, the POI is considered to be a restricted person with respect to the state border, but is not considered to be a restricted person with respect to the city border.

Embodiments of the boundary crossing management system seek to identify when a person of interest is attempting to cross a boundary for which the person is considered a restricted person. The identification of an attempted restricted boundary crossing may allow the physical prevention of the restricted person crossing the boundary.

### Boundaries

A boundary defines a physical region or locality. A boundary may be the perimeter of a region, a partial perimeter, or may be an ingress or egress point for a region. Examples of a boundary include: a state, country or regional border or part there-of; a venue, building, campus or other location; an educational, medical, entertainment or sporting campus; a declared hotspot region, or region of restricted access.

A boundary may be defined in terms of a physical location or region. The physical location or region may be defined in terms of a two-dimensional area, or a three dimensional area including a height parameter. A boundary may also be defined in terms of a time period. For example, in some embodiments, boundary crossing management is performed for a boundary, only during a defined time period or periods.

The boundary crossing management method and system, described herein, seeks to identify when a person who is attempting to cross a boundary, to enter or exit a region defined by the boundary, is a person that is restricted from crossing the boundary, i.e. a restricted person. For brevity, in describing the boundary crossing management system, this disclosure may refer to the boundary crossing management system as detecting when a restricted person is attempting to cross a boundary to enter a region; however, it is to be understood that the boundary crossing management solution may also be applied, using the techniques described herein, to detect when a restricted person is attempting to cross a boundary to exit a region defined by that boundary.

### Boundary Crossing Restrictions

In some situations a restricted person may be inclined to attempt to circumvent the movement restriction measures, and attempt to cross a boundary for which they are restricted. For example, a person that is subject to a quarantine order on medical grounds may attempt to enter an entertainment venue. This would be undesirable from the point of view of the DMA.

Embodiments of the disclosure provide a system and method to identify whether a person who is attempting to cross a boundary at a checkpoint (otherwise known as a checkpoint user) to enter or exit a region is a restricted person, for the purposes of that region, in accordance with the requirements of the DMA.

### Checkpoints

A boundary may comprise one or more checkpoints, which are ingress points for persons to enter the region defined by the boundary. In some embodiments, the checkpoints may also serve as egress points.

Embodiments of the present disclosure locate, at the checkpoints, a checkpoint management system, which works in conjunction with server 102 to perform the boundary management process.

The checkpoint management system collects the name of the checkpoint user and collects name verification information, which can be used to provide a level of verification that the name collected is the actual name of the person.

The checkpoint management system provides the name and the name verification information to the server 102. The server 102 determines, based on the name and the name verification information, whether the checkpoint user is a restricted person with respect to that boundary. The server 102 provides a permission outcome of the determination to the checkpoint management system, and based on that permission outcome, the checkpoint management system indicates whether the person is permitted to cross the boundary. The permission outcome may be a positive permission outcome, indicating that the checkpoint user is permitted to cross the boundary, or a negative permission outcome, indicating the checkpoint user is not permitted to cross the boundary.

In some embodiments, the checkpoint management solution comprises a person in authority at the checkpoint, who can view photo identification of a checkpoint user. The person in authority can verify that the photograph on the photo identification sufficiently matches the checkpoint user. The person in authority can provide the name of the checkpoint user to the server 102, via a web interface, application interface or other communication means.

In another embodiment, the checkpoint management system comprises a checkpoint management device. The checkpoint management device comprises a processor, a screen and a camera, and is communicatively coupled to the server 102. The checkpoint management device obtains the name of the checkpoint user via a user interface, or via the camera scanning an identification card provided by the person.

Using facial recognition and comparison processes, the checkpoint management device determines whether the person presenting the photo identification matches the facial photo provided on the photo identification. Alternatively, the checkpoint management device scans the photo identification, and takes a photo of the checkpoint user, and sends both the scan and the photo to the server 102 for facial recognition and comparison processes.

In one embodiment, the checkpoint management device is communicatively coupled to physical barriers at the checkpoint. The physical barriers operated in response to commands provided by the checkpoint management device. Accordingly, when the checkpoint management device receives an indication from the server 102 that the checkpoint user is not a restricted person, the checkpoint management device sends an ‘open’ command to the physical barriers, which open in response to allow the person to cross the checkpoint. Alternatively, when the checkpoint management device receives an indication from the server 102 that the checkpoint user is a restricted person, the checkpoint management device does not send an ‘open’ command to the physical barriers.

The checkpoint management device further comprises one or more biometric sensors. The sensors collect biometric data, such as fingerprint, retina, voice or face recognition. The biometric data is collected by the checkpoint management in the event of an Amber Path Validation, as described below.

### Pool of Restricted Names

The DMA maintains a list of names of restricted persons. This is called the pool of restricted names. Each name in the pool of restricted names may be associated with other identifying attributes that identify the unique identity of the restricted person. For example, each entry in the pool of restricted names may be associated with a birth date, residential address, reference biometric data or other identifying information.

In some embodiments, the DMA maintains pool of restricted names for each boundary. In some embodiments, the DMA maintains a list of restricted persons, each associated with a region of restriction. A set of restricted persons may be associated with a boundary, such that persons in that set of restricted persons are not permitted to cross that boundary. Alternatively a restricted person may be associated with a plurality of boundaries, such that the restricted person is not permitted to cross any of the plurality of boundaries. For brevity, this disclosure will describe a set of restricted persons being associated with a boundary, however it is to be understood that one or more restricted persons in that set of restricted persons may be restricted from crossing other boundaries as well.

### Green Path and Amber Path Validations

The boundary crossing management solution comprises two validation paths to validate whether the checkpoint user is permitted to cross a boundary, and is therefore not a restricted person for that boundary.

Firstly, the server 102 performs the green path validation to determine whether the name provided by the checkpoint user, is included in the list of restricted names associated with that boundary. If the name provided by the person is not included in the list of restricted names associated with that boundary, the server 102 determines that the person is permitted to cross the boundary.

If the name provided by the person is included in the list of restricted names associated with that boundary, the server 102 determines whether the checkpoint user is a restricted person, or whether the checkpoint user merely shares the same name as a restricted person. To make this determination, the server 102 performs the amber path validation process. The amber path validation involves obtaining biometric data from the checkpoint user, and comparing the biometric data to reference biometric data of restricted persons with the same name as the checkpoint user.

An amber validation path may result in the person being permitted to cross the boundary, if it is determined that the person is not a restricted person, or may result in a High Level Action (HLA) if the person appears to be a restricted person, based on the amber path validation process.

A HLA is an alert message provided by the server 102, to the Enforcing Authority (EA) at the completion of the amber path validation process.

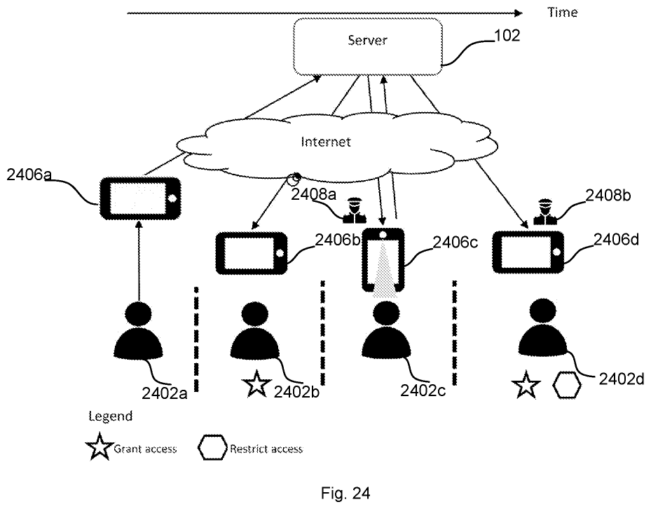

### FIG. 24—Checkpoint Management

FIG. 24 illustrates boundary crossing management at a checkpoint, according to an embodiment. A checkpoint user 2402 provides a name to the checkpoint management system. In the embodiment illustrated in FIG. 24, the checkpoint management system comprises a checkpoint management device which is indicated, at different points in time, by reference numerals 2406a-d. In other embodiments, the checkpoint management system may comprise one or more checkpoint persons.

The checkpoint user 2402 may provide the name 2404 to the checkpoint management device 2406a verbally, or via typing the name into the device 2406a, or by using the device 2406 to scan an identification card, such as a driver's license.

The checkpoint management device 2406a relays the name provided by the checkpoint user 2402a to the DMA operating on server 102. In situations in which the checkpoint user 2402a used the checkpoint management device 2406a to scan an identification card, the checkpoint management device 2406a may provide the scanned image to the server 102, and the server 102 may process the scanned image to determine the name provided on the identification card.

The server 102 determines, based on the provided name, the pool of restricted names and other factors, whether green path validation is applicable for this boundary crossing. If green path validation is applicable, the server 102 communicates an ‘access granted’ signal to the checkpoint management device 2406b. The checkpoint user 2402b (same as checkpoint user 2402a) is permitted to cross the boundary.

If the amber path validation is applicable, the server 102 requests that the checkpoint management device 2406c obtain biometric data from the checkpoint user 2402c (same as checkpoint user 2402a). Biometric data, such as a fingerprint, face identification attributes, or other biometric data, is obtained from the checkpoint user 2402c by the checkpoint management device 2406c.

In another embodiment, the checkpoint management device 2406c provides an indication to the checkpoint user 2402c that the server 102 has requested biometric data; however, the checkpoint management device 2406c does not obtain the biometric data. Instead, the biometric data can be obtained by a device associated with the checkpoint user 2402c, such as the person's mobile communication device. This approach may be more desirable in situations where privacy concerns make it undesirable for the checkpoint management device to obtain biometric data associated with the checkpoint user 2402c.

The biometric data obtained from checkpoint user 2402c is sent to server 102. Server 102 compares the biometric data obtained with one or more reference biometric data entries stored in the server 102 and associated with the name provided by checkpoint user 2402a.

If the server determines that the biometric data obtained from checkpoint user 2402c is associated with a restricted person listed in the server's list of restricted persons for this boundary, the server 102 sends an ‘access denied’ message to the checkpoint management device 2406d. In response to receiving an ‘access denied message’, the checkpoint management device 2406d displays an indication that access has been denied for the checkpoint user to cross the boundary. If the checkpoint management device 2406d is operably connected to a physical barrier, the checkpoint management device 2406d sends a signal to close, or keep closed, the physical barrier to inhibit the checkpoint user from crossing the boundary.

If the server determines that the biometric data obtained from checkpoint user 2402c is not associated with any person listed as a restricted person in the server's list of restricted persons for this boundary, the server 102 sends an ‘access permitted’ message to the checkpoint management device 2406d. In response to receiving an ‘access permitted message’, the checkpoint management device 2406d displays an indication that access has been permitted for the person to cross the boundary. If the checkpoint management device 2406d is operably connected to a physical barrier, the checkpoint management device 2406d sends a signal to open the physical barrier to allow the checkpoint user to cross the boundary.

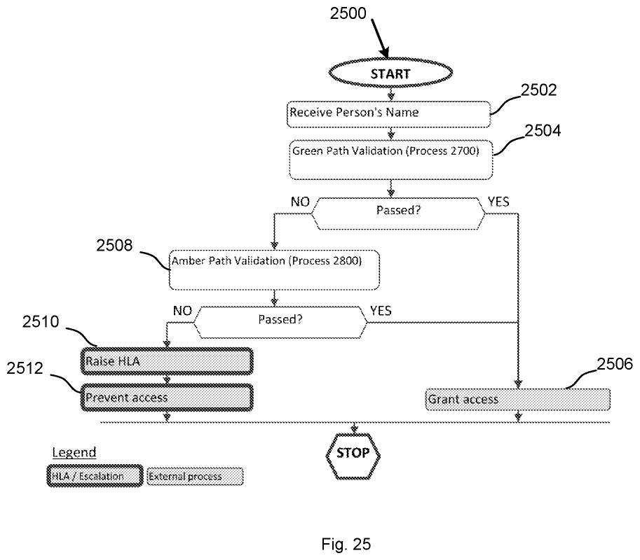

### FIG. 25—Checkpoint Management Process

FIG. 25 is a flowchart illustrating the checkpoint management process 2500 as performed by the server 102 in conjunction with the checkpoint management device, according to an embodiment.

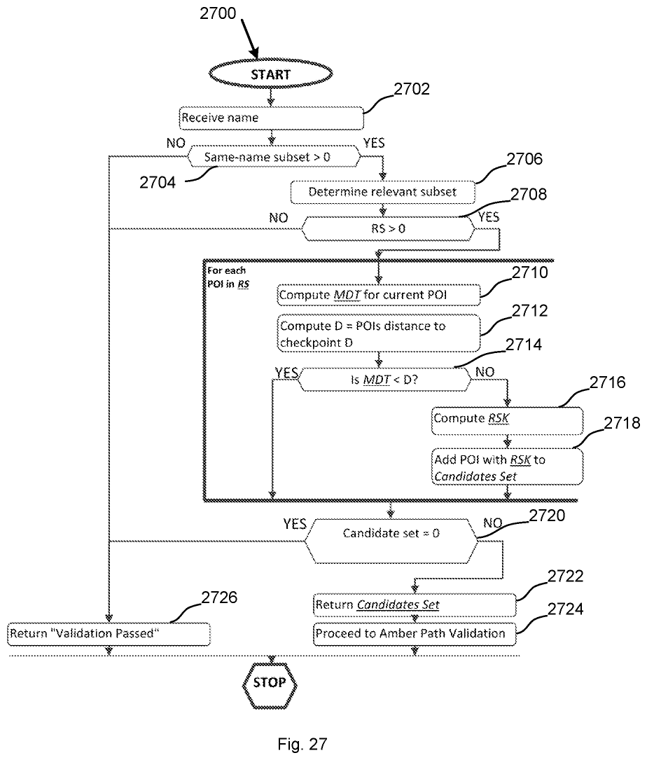

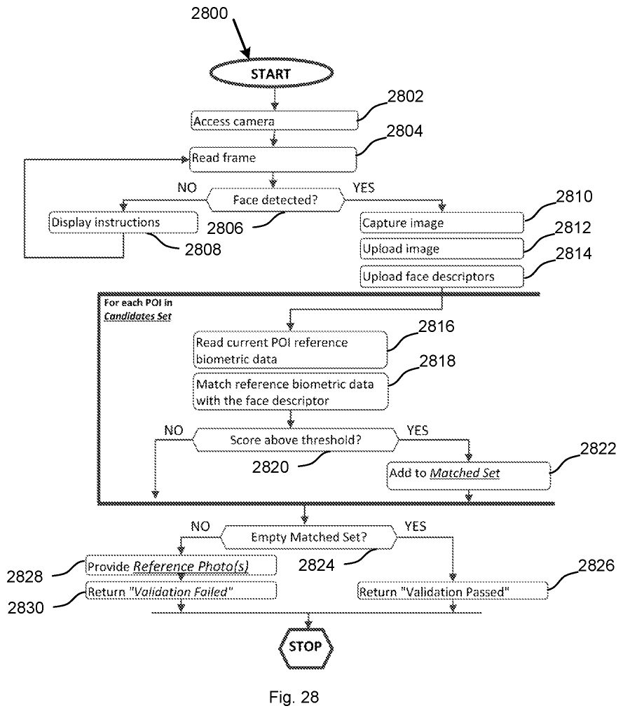

In step 2502, the server 102 receives, from the checkpoint management device, the name of a checkpoint user. In step 2504, the server 102 performs the green path validation process, which is described below in relation to FIG. 27. If the green path validation process 2700 passed, in step 2506 the server 102 sends a positive permission outcome message to the checkpoint management device indicated that access is permitted for the checkpoint user to cross the boundary. If the green path validation process 2700 did not pass, in step 2508 the server 102 performs the amber path validation process, which is described below in relation to FIG. 28.

If the amber path validation process 2800 passed, in step 2506 the server 102 sends a positive permission outcome message to the checkpoint management device indicating that access is permitted for the checkpoint user. If the amber path validation process 2800 did not pass, in step 2510 the server 102 raises a High Level Alert (HLA) to the DMA, and in step 2512 the server 102 sends a negative permission outcome message to the checkpoint management device indicating that access is not permitted for the checkpoint user to cross the boundary.

### Enforcement

The permitting or denying of a boundary crossing can be managed by an enforcing authority at the boundary checkpoint. In an embodiment, both the EA and the checkpoint crews have adequate processes in place. Depending on circumstances, both EA and checkpoints crew can be involved in these processes or just the EA. An example involvement of EA without checkpoint crew would be a small local event where the organisers would not have the capacity to prevent access so the HLA is issued at the EA which will deploy on site to address the violation.

### Optimising Green Path Validation

In one embodiment, the Green Path Validation (GPV) process is optimised to produce accurate decisions even for commonly used names.

When the pool of restricted names is large, there is an increased probability that there is a duplication of one or more names in the pool of names. For example, common names may be included in the pool of restricted names multiple times. Accordingly, in situations where a checkpoint user has a name that occurs more than once in the pool of restricted names, there is a need to determine whether the checkpoint user is a restricted person who is not permitted to cross the boundary, or whether the checkpoint user is a non-restricted person.

The boundary crossing management solution can be optimised, as described below, such that a prompt decision can be made, in the majority of cases, including for common names, to enable a fast transfer through the boundary checkpoint and to avoid unnecessary complications for unrestricted persons.

Advantageously, the optimisation techniques described below require no additional data to be provided from the checkpoint management system to the DMA.

### Set Reduction Algorithm

In large scale applications, the probability for persons with statistically common names to be listed as restricted persons may be high. Similarly, the probability that a person with a common name becomes a checkpoint user at some point is also high. In some embodiments, the server implements a set reduction algorithm, which aims to achieve a fast reduction of the number of restricted persons for the amber path validation process, for persons with common names.

In one embodiment, the green path validation process includes a set reduction algorithm that determines a candidate subset of restricted persons associated with a boundary checkpoint, wherein the candidate subset is a subset of the set of restricted persons. The candidate subset comprises restricted persons with the same name as a checkpoint user, and with a reasonable possibility of physically being at the boundary checkpoint. This reasonable possibility is a level of likelihood that can be configured, as detailed below.

The server determines the candidate subset based on which of the restricted persons, associated with a boundary, and sharing the same name a checkpoint user, could be physically present at the boundary checkpoint, based on whether the distance between the last confirmed location of the restricted person and the checkpoint is within the range of distance that can physically covered in the time since the last location confirmation for that restricted person.

A purpose of determining the candidate subset is to speed up the process of boundary crossing management by decreasing the number of times the amber path validation process needs to be performed. Reducing the number of times the amber path validation process is performed is desirable because the amber path validation involves the collection of biometric data from checkpoint users. The collection of biometric data takes time and may be considered intrusive.

By performing the location confirmation process for a restricted person, the server 102 is aware of the last confirmed location and time of last location confirmation for the restricted person. Accordingly, the server 102 can determine whether it is possible (within parameters of probability defined for the set reduction algorithm) that the restricted person is physically present at the boundary. If the restricted person is unlikely to be physically present at the boundary, the restricted person can be removed from the candidate subset of restricted persons.

In one embodiment, in a first step the set reduction algorithm determines the relevant subset of restricted persons for a boundary checkpoint, for a particular name, based on the Maximum Check Interval and the Maximum Travel Speed of restricted persons associated with that boundary checkpoint and having that particular name.

### Maximum Check Interval

The Maximum Check Interval (MCI) is a measurement of time that represents the maximum time period between two location confirmation checks for a restricted person (i.e. a POI). The MCI may be derived from the monitoring intensity.

### Maximum Travel Speed

The Maximum Travel Speed (MTS) is a measure of speed that represents an expected maximum speed at which a restricted person (i.e. a POI) could travel. A high MTS may be set for a restricted person, for example 1,000 km/h, but as detailed below, a high MTS would increase the validation radius.

In one embodiment, a practical value of the MTS is 120 km/h or 2 km/minute. For this value, the maximum average speed achievable by a POI travelling from their required location to the location of a boundary checkpoint in a normal urban landscape is 120 km/h.

### Validation Radius

The Validation Radius (VR) is a measure of distance from a boundary checkpoint, defining the maximum distance from the checkpoint for which it can be assumed that a POI could have travelled to arrive at the boundary checkpoint at a particular time.

The server 102 determines the VR based on the MTS and the MCI for a restricted person, as shown below:

VR=MTS*MCI (1)

In one example, the server 102 determines that a restricted person cannot have travelled further than VR=480 km to the checkpoint location if MTS=120 km/h and MCI=4 hours.

In one embodiment, the server 102 determines the validation radius as straight radial lines which define a circular region. In other works, the server 102 determines the validation radius in terms of a distance “as the crow flies”. In another embodiment, the server 102 determines multiple validation radii in multiple directions, taking into consideration available road networks, and the validation radii define a non-circular area.

### Determining the Relevant Subset

The server 102 determines a relevant subset of restricted persons for a boundary checkpoint. The relevant subset being a subset of the whole set of restricted persons associated with a boundary, wherein each restricted person in the subset has the same name and wherein the last confirmed location for each person is within the validation radius centred on the boundary checkpoint.

### Determining the Candidate Subset

For each person in the relevant subset, the set reduction algorithm determines the potential Maximum Distance Travelled (MDT). The MDT represents the maximum distance that a POI could have travelled from the POI's last confirmed location, travelling at the MTS associated with the POI, if travel started right after the last location confirmation.

The value of the MDT is zero at the time of a POI's location confirmation, and increases linearly with the time after the location confirmation.

MDT=Interval(Current time,Last confirmed location timestamp)*MTS (2)

The server 102 applies the set reduction algorithm, as part of the green path validation process 2700. The server 102 calculates the MDT for each person in the relevant subset to determine whether that person should be included in the candidate subset for an amber path validation.

If the MDT for a person in the relevant subset is less than the distance between the required location of the POI and the location of the boundary checkpoint, then the server 102 can surmise that the POI could not have travelled to the boundary checkpoint at this point in time, and therefore the checkpoint user is not the POI, but merely shares the same name as the POI. Accordingly, that POI need not be included in the candidate subset of restricted persons.

if MDT<Distance(Quarantine location,Checkpoint location),then POI is not a candidate (3)

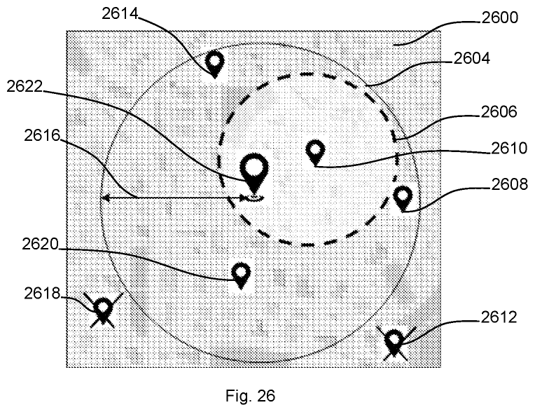

### FIG. 26—Set Reduction Algorithm Example

FIG. 26 is a diagram illustrating a map of a region 2600, a location of a boundary checkpoint 2622, last confirmed locations (2610, 2608, 2612, 2614, 2618 and 2620) of persons in the same-name subset of restricted persons associated with the boundary checkpoint 2622, according to an embodiment.

The server 102 calculates the validation radius 2616, which defines a validation boundary 2604 centred on the boundary checkpoint 2622.

In the first level of filtering, the server 102 determines that POIs associated with locations 2612 and 2618 are outside the validation boundary 2604, according the server 102 removes POIs associated with locations 2612 and 2618 from the relevant subset. Accordingly, the relevant subset comprises POIs associated with location 2608, 2610, 2614 and 2620.

In the second level of filtering, the server 102 determines the MDT for the POIs associated with each of locations 2608, 2610, 2614 and 2620. In the example illustrated in FIG. 26, the server 102 determines that the MDT for each POI associated with locations 2608, 2614 and 2620 is less than the distance from that location to the boundary checkpoint 2622. Accordingly, the candidate subset comprises only the POI associated with location 2610. The server 102 may perform amber path validation to determine whether the person at checkpoint 2622 is the POI associated with location 2610.

### Risk Level

The system computes a risk level (RSK) that models the probability of success for a fraud attempt. The actual probability of a restricted person attempting fraud depends mostly personality traits. The risk factor computed here quantifies the probability fraud to be executed irrespective of personal traits.

In this case, execution of fraud is a restricted person presenting at the checkpoint. The probability that fraud is executed will be higher for closer venues. For example, a restricted person attempting to fraudulently enter a pub close to their quarantine location. By contrast attempting to watch a game 50 km away from a restricted person's required location is less likely to occur.

The server determines the Risk Level, RSK, as a percentage with 100% being the highest risk for fraud execution.

$$RSK = ( 1 - \frac{VR - Distance ( Quarantine location , Checkpoint location )}{VR} ) * 100 ( \% ) \qquad ( 4 )$$

The RSK allows targeting of the operational options such as escalation paths.

### FIG. 27—Green Path Validation Process

FIG. 27 is a flowchart illustrating the green path validation process 2700, as performed by the server 102 in conjunction with a checkpoint management device, according to an embodiment.

In step 2702, the server 102 receives, from a checkpoint management device located at a boundary checkpoint, the name of a checkpoint user. In step 2704, the server 102 determines the set of restricted persons associated with the boundary, and determines the same-name subset of restricted persons that have the same name as the checkpoint user.

If the same-name subset is empty (i.e. there are no restricted persons with the same name as the checkpoint user), the server 102 completes the green path validation process by indicating 2726 that the boundary crossing is permitted.

If the same-name subset is not empty, the server 102 proceeds to step 2706, in which the server determines the validation radius and determines the relevant subset of restricted persons whose last confirmed location is within the validation radius. The relevant subset of restricted persons is a subset of the same-name subset of restricted persons.

If the relevant subset is empty, the server 102 completes the green path validation process by indicating 2726 that the boundary crossing is permitted.

If the relevant subset is not empty, the performs a set of operations for each POI in the relevant subset, to determine whether that person should be included in the candidate subset. More specifically, in step 2710, the server 102 computes the maximum distance travelled for a POI, and in step 2712, the server 102 computes the distance between the POI's last confirmed location and the location of the boundary checkpoint.

In step 2714, if the maximum distanced travelled is less than the distance between the POI'S last confirmed location and the location of the boundary checkpoint, the server 102 includes the POI in the candidate subset. Otherwise, the server 102 proceeds to step 2716, in which the server 102 determines RSK. In step 2718, the server 102 includes a POI who has a RSK in the candidate subset.

In step 2720, the server 102 determines whether the candidate subset, as determined in looped steps 2710 through 2718, is empty. If the candidate subset is empty, the server 102 completes the green path validation process by indicating 2726 that the boundary crossing is permitted. However, if the candidate subset is not empty, the server 102 completes the green path validation process by proceeding 2724 to the amber path validation process 2800.

### FIG. 28—Amber Path Validation Process

FIG. 28 is a flowchart illustrating the amber path validation process 2800, wherein the biometric data obtained and compared to perform the validation is face detection data, according to an embodiment. Steps 2802 through 2814 are performed by a checkpoint management device located at a boundary checkpoint. Steps 2816 through 2830 are performed by server 102.

In step 2802, the checkpoint management device accesses the device's camera to take a photo of the checkpoint user's face. In step 2810, the checkpoint management device takes the photo, and in steps 2812 and 2814 the checkpoint management device uploads the image and face descriptors.

### System Self-Tuning

In some embodiments, the location confirmation and boundary crossing management system includes a self-tuning mechanism. The purpose of the system self-tuning is to maximize the number of successful Green Path Validations (GPV). After entering the name, a successful Green Path Violation (GPV) enables the member of the public to simply walk out of the checkpoint. This is desirable in cases where there is a need to process large volumes public numbers such as sports events. By comparison, a failed GPV triggers the Amber Path Validation (APV) that includes a bio-metric check. Obviously APV slows the checkpoint operation.

The self-tuning methods described here enhance the ability of the GPV to deliver “Passed” results, (e.g. return with an empty candidate subset) in as many cases as possible, or produce the smallest safe candidate subset.

The system self-tuning comprises methods to improve the GPV performance by adjusting parameters of the whole system, and performing actions such as triggering additional location confirmation checks.

The self-tuning aspect refers to adjusting the Monitoring Profile of a certain POI from a purely random approach to a quasi-random pattern adjusted to discourage fraud attempts and to detect early such attempts.

The self-tuning logic takes into account the Monitoring Intensity and Maximum Check Interval (MCI), as well as the checkpoints relevant at any point in time. For example, it will adjust automatically during a weekend when temporary checkpoint (i.e. checkpoints for sporting or entertainment venues) may be in place.

The optimisation uses a few dimensions such as time, checkpoint location, statistics on persons' names and checkpoint volumes. By using this information, the server can trigger additional location confirmation checks at certain times, in certain areas for persons with common names.

The method described here operates across the quarantine monitoring and border/boundary crossing management system areas in an integrated manner. It provides self-tuning access optimisation and separation of roles between EA and checkpoint crews.

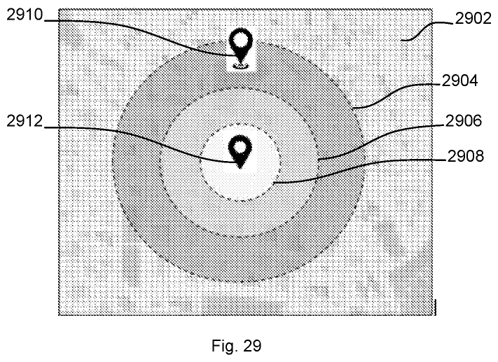

FIG. 29—Minimising the Candidate Subset

The purpose of the system self-tuning described here is to minimise the candidate subset for a boundary checkpoint and name. The candidate subset is the smallest safe subset of POIs that need to be compared against in an Amber Path validation. The most optimal candidate subset is an empty one. This can happen in a hypothetical scenario in which the system has just performed location checks on all POI having the same name as the person at the checkpoint. In such a case, no matter how common the name is, the candidate subset will be empty as having their location checked, none of them could attend any venue fraudulently.

The first step in understanding the ways in which the algorithm can be optimised is to analyse what happens to the Maximum Distance Travelled (MDT) in time. MDT is zero at the moment of a successful location check. It starts growing with time in a linear fashion.

FIG. 29 illustrates the change in the area represented by a POI's MDT, as the MDT increases over time, according to an embodiment. The POI'S required location 2912 is also the last confirmed location of the POI. A boundary checkpoint 2910 is located at a distance from the POI's last confirmed location 2912. At a time T1 from the time of last confirming the POI'S location, the area in which the POI could have travelled, based on the MDT at time T1 is enclosed by perimeter 2908. Similarly, at a time T2, later than T1, from the time of last confirming the POI's location, the area in which the POI could have travelled, is enclosed by perimeter 2906. At either of times T1 or T2, the POI would not be included in a candidate subset associated with checkpoint 2910, because the POI is deemed to not have been able to travel to the checkpoint 2910 by that time.

At time T3, from the time of last confirming the POI's location, the area in which the POI could have travelled, based on the MDT at time T3 is enclosed by perimeter 2904. Checkpoint 2910 is situated within perimeter 2904. Accordingly, at time T3, the POI would be included in a candidate subset associated with checkpoint 2910.

Accordingly, after a certain time, MDT becomes larger than the distance between the last confirmed location of a POI and a checkpoint. After this point in time the POI may be included in the candidate subset, as the POI has the potential to attempt to cross the boundary.

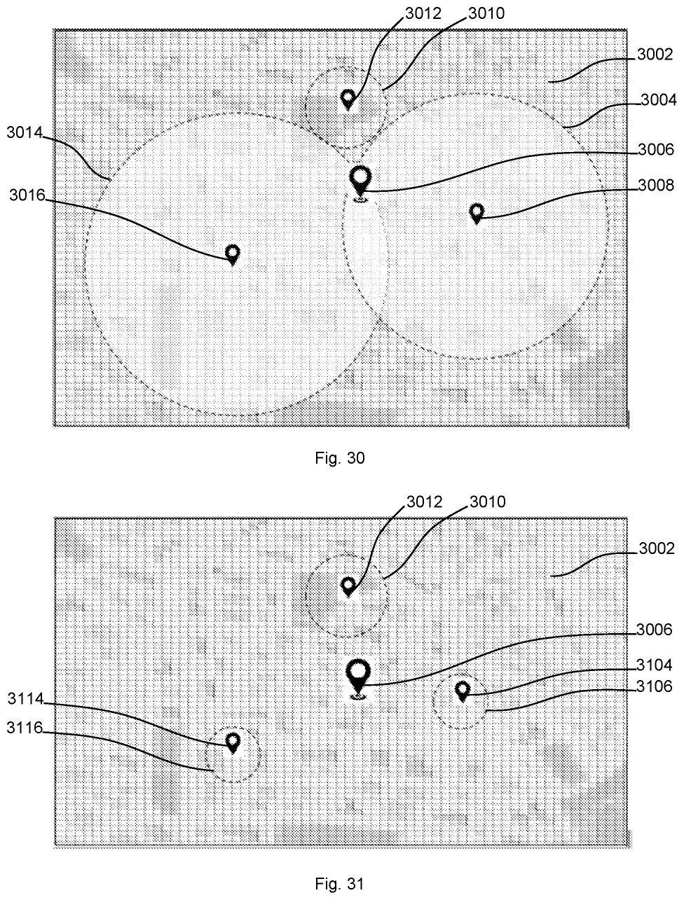

### FIG. 30—Distant POIs Included in the Candidate Subset

The MDT is calculated as a function of, at least, the maximum travel speed of the POI and the time since the last location confirmation. Accordingly, the MDT can differ between two respective POIs due to one POI having a lower maximum travel speed compared to the other POI. Furthermore, the MDT can differ between two respective POIs due to one POI having successfully completed a location confirmation more recently than the other POI.

FIG. 30 illustrates areas in which POIs could have travelled at a point in time, based on each POI's MDT, according to an embodiment. FIG. 30 illustrates that POIs located more distantly from a checkpoint may be included within the candidate subset, although more locally located POIs are not included within the candidate subset. Checkpoint 3006 is located within area 3002. A first POI has a last confirmed location of 3016, and the area in which the first POI could have travelled, based on the first POI's MDT is enclosed by perimeter 3014. Notably, checkpoint 3006 is within perimeter 3014, and therefore the first POI may be included in the candidate subset.

Similarly, a second POI has a last confirmed location of 3008, and the area in which the second POI could have travelled, based on the second POI's MDT is enclosed by perimeter 3004. Notably, checkpoint 3006 is within perimeter 3004, and therefore the second POI may be included in the candidate subset.

A third POI has a last confirmed location of 3012. Accordingly, the area in which the third POI could have travelled, based on the third POI's MDT is enclosed by perimeter 3010. Checkpoint 3006 is not within perimeter 3010, and therefore the third POI is not included in the candidate subset.

Based on the scenario illustrated in FIG. 30, the candidate subset comprises the first and the second POIs.

### FIG. 31—Additional Location Checks to Reduce Candidate Subset

One method in which to reduce the candidate subset and to optimise the GPV effectiveness is to maintain MDT as low as possible by increasing the frequency of location checks.

FIG. 31 illustrates areas in which POIs could have travelled at a point in time, based on each POI'S MDT, after server 102 performs additional location confirmation checks for the first and second POIs, according to an embodiment.

FIG. 31 illustrates the location of the checkpoint 3006, and the last confirmed location of the third POI 3012 as illustrated in FIG. 30. To reduce the MDT for the first and second POIs, the server 102 performs another location confirmation check for each of the first and second POIs. As a result, location 3114 represents the most recent confirmed location of the first POI, and location 3104 represents the most recent confirmed location of the second POI.

The time since the last confirmation check for the first and second POIs has been reduced, when compared to the scenario illustrated in FIG. 30. Accordingly, the area in which the first POI could have travelled, based on the first POI's MDT is enclosed by perimeter 3116, which is smaller than the area enclosed by perimeter 3014. Similarly, for the second POI, the area enclosed by perimeter 3106 is smaller than the area enclosed by perimeter 3004.

The checkpoint 3006 is enclosed by none of areas 3010, 3116 or 3106. Accordingly, the candidate subset for checkpoint 3006 does not include any of the first, second or third POIs.

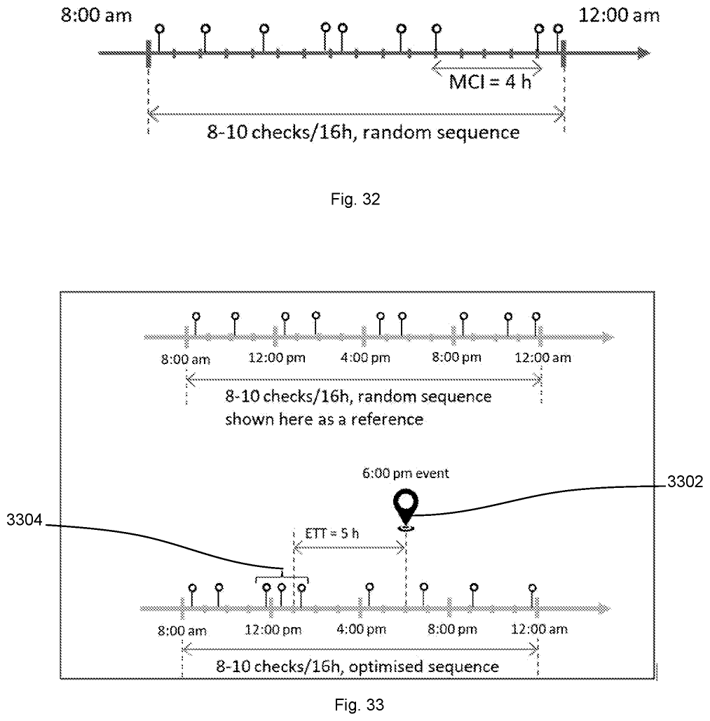

### FIG. 32—Frequency of Location Checks

The relevant system parameters that control the frequency of location checks are monitoring intensity and monitoring profile. The monitoring intensity specifies the number of location checks to be performed over a period of time. A higher intensity will involve a larger number of checks. The monitoring profile is a schedule of changes to monitoring intensity over a period of time.

FIG. 32 displays a monitoring profile, for a POI, covering 8:00 am to 12:00 am with 8 to 10 checks shown, according to an embodiment. The maximum check interval (MCI) is 4 h and an instance of the maximum check interval occurred between 7:00-11:00 pm.

The monitoring profile, for a POI, can be optimised by ensuring a higher density of checks around critical times during the monitoring times, while maintaining the prescribed monitoring intensity and fitting within the MCI i.e. gaps no longer than 4 hours, in the example illustrated in FIG. 32.

This optimisation will ensure minimal MDTs at critical time while allowing the MDT to grow at other times.

### Common Names Subset

The system self-tuning needs to happen only for common names. The reason for that is that for names that are un-common in that area the candidate subset will be empty or small. As a result, there is no need to perform optimisation to reduce the candidate subset. A name is included in the subset of common names if its representation in the pool of restricted persons is higher than a threshold in a particular geographical region. The threshold value can be static or dynamic.

In one embodiment, the server utilises machine learning techniques to optimise the threshold valid. For each name in the subset of common names, two sets of attributes are provided to allow a machine learning based optimisation of the threshold value. The first set of attributes comprises geographical/location attributes, such as equal areas, equal population, national administrative areas like local government areas and councils. The second set of attributes comprises POI additional attributes, such as demographic data (age group, gender etc.)

The attributes used by the server to optimise the threshold value may depend on what data is available to the Decision-Making Authority (DMA) at POI level

The server utilises the two sets of attributes to optimise the threshold valid in two ways. Firstly, at the initial setup, lower thresholds can be set in certain geographical areas or for particular cohorts. Secondly, the server may perform a continuous adjustment to optimises the value for the threshold based on past information. Past information can include data about fraud attempts attributes as well as reliability of geo-location checks. An example in this last category can be the fact that POIs in the age group 20 to 35 require more repeats of the location checks if they are considered not reliable from the point of view of location checks than other age groups. Another example can be the fact that most fraud attempts are committed in particular metro areas by say males in the 20-35 age group.

For the a common name set, the system will have different thresholds that are continuously adjusted as described above. Critical areas will have a lower threshold. In this way a sporting event can be classified as relevant in one geographical area while a similar event might not be classified as relevant in other areas.

Additionally names can be added to the set of common names if the DMA decides to do so.

### Checkpoint Profiling

In one embodiment, the server performs checkpoint profiling. The purpose of checkpoint profiling is to rank checkpoints from the point of view of optimisation of the GPV. Additionally, the checkpoint profiling data assists with providing information about the risk of attracting fraudulent behaviour, understanding which optimisation methods to use and helping with escalation response.

For example, a boundary crossing with continuous traffic will offer less options for optimisation than say a one-off sporting event with surge traffic, despite both having a similar “attractiveness” for fraudulent behaviour. In the case of the sporting event there is a well-defined timeframe for the surge traffic and this aspect can be used in optimising the monitoring within given monitoring intensity and monitoring profiles.

Checkpoint profiling uses two levels of weighting that can be refined to reflect the need of including venues in the optimisation. The weighting scores start with default values and then self-adjust based on historical data. However, the system can be forced to use pre-set scores so that focus on certain types of venues can enhanced on demand. The scores can reflect the nature of various areas in which the checkpoints are located.

Checkpoints may be classified based on dimensions. Each checkpoint classification dimension carries a weight from the perspective of GPV the optimisation. In addition, individual scores help rank the checkpoints from different perspectives such as need to have EA resources nearby.

The list of checkpoint profiling dimensions with starting sample scores can include:
Type of checkpoint (10%)
Traffic volume size (15%)
Types of public access (15%)
Pull risk level (30%)
Preparedness level (30%)

Disclosed below is a non-exhaustive list of examples of defining and interpreting the checkpoint profiling dimensions mentioned. The order of the values in each dimension is based on the ability of that point to contribute to optimisations, with the first value being the one with the biggest contribution. Each value in a dimension gets a score that needs to reflect its impact from the point of view of that dimension. Using the example of the pull risk level the following scores, out of 10, could be used:
High—score 10 out of 10.
Unknown—score 8
Low—2

An “Unknown” pull risk level is not scored as 5 but as 8. This means that to be on the safe side, the system should treat the “Unknown” closer to the way it handles “High”.

Based on boundary checkpoint types the following classification exists:
Temporary—for instance sporting events that can be treated as on-off despite the actual venue being a permanent structure.
Permanent—border crossings, sport venues, art galleries, pubs, shopping centres

Temporary events can contribute more effectively to the optimisation as they provide a time interval to focus on.

Based on the size of traffic volumes, regions associated with boundaries can be characterised according to the number of persons within, or expected to be within the boundary, as:
Very Large—say over 5,000 participants
Large—say 1000-5,000 participants
Medium—say 200-1,000 participants
Small—say under 200 participants

The participant ranges used above are examples only. The participant ranges should also be different for different types of areas, rural versus urban, and different population densities.

Based on the type of public access, checkpoints can be characterised as:
Surge—typical example large sport events or concerts. In this case, a boundary checkpoint is closed for most of the time except for the duration of particular events when the traffic reaching very high volumes.
Periodic—a periodic boundary checkpoint has low traffic or is closed for most of the time with the exception of certain time intervals when high volumes occur. An example of a periodic checkpoint is a pub or restaurant.
Continuous—a continuous checkpoint will have a somewhat continuous low-level of traffic. An example of a continuous checkpoint may be a regional border crossing. A checkpoint may be considered a continuous checkpoint if the total time when the traffic is under a certain threshold is not be more than a certain percent of the measured time, say daily. As an example, an airport has continuous access if traffic does not drop under 10% of peak for more than 6 hours per day (25% of time).

Based on the pull risk levels, checkpoints can be characterised as:
High—for events that can attract more fraudulent behaviour.
Unknown
Low

Note that the pull risk is not a reflection of the type and quality of various events or venues. It is rather a linked to profiling of persons likely to attend the venue or event associated with the checkpoint.

Based on the Preparedness level, checkpoints can be characterised as:
Low—one-off events or events with volunteer crews, such as local weekend sporting events.
High—organised checkpoints with unevenly trained crews, such as pubs or stadiums.
Strict—highly organised checkpoints with highly trained crews, such as border crossing or airport.

Checkpoints with a Low Preparedness level may need more assistance in fraud prevention.

A checkpoint gets a general score based on the weighted scores on the profiling dimensions in order to establish its priority in the optimisation and dimension-based scores. Dimension based scores help rank the checkpoints from different perspectives such as the need to have EA resources nearby.

For instance, a motor bikers' rally can be described as:
Type of checkpoint—Temporary
Traffic volume size—High
Types of public access—Surge
Pull risk level—High
Preparedness level—Low— volunteer-based crews

As such, the rally may be considered a prime target to be included in the optimisation mechanism

### Relevant Checkpoint Subset (RCS)

Relevant Checkpoint Subset (RCS) includes those checkpoints that are above the threshold score within a POI's validation radius.

In addition, the combination of max scores on Pull risk level and high score on Traffic volume should flag the need to have EA resources nearby, as the venue while likely to experience fraud attempts will not have the capacity to cope with restricting access in such instances.

### Optimisation Process

In one embodiment, the server 102 performs an optimisation process, to optimise boundary crossing management. The optimisation process adjusts the monitoring intensity for POIs with names in the Common Name Subset to optimise the process of boundary crossing management. The optimisation process does this by taking into account the checkpoints/events that happen in the designated area.

As noted with reference to FIG. 29, as time passes, area of potential travel around a POI, as defined by the MDT, increases. At some time, the area may include a checkpoint. At that time, the POI may be added to the Candidate Subset produced by the GPV if a person with the same name as the POI attempts to cross at boundary at that checkpoint.

To address this situation the system will optimise the monitoring Profile of the POI's matching the Common Names Subset as shown below to reset the MDT.

FIG. 33—Monitoring Profile with Scheduled Event

FIG. 33 displays a monitoring profile, for a POI, covering 8:00 am to 12:00 am with 8 to 10 checks shown, according to an embodiment. An event 3302, associated with a boundary checkpoint, is scheduled to start at 6 pm. The Estimated Travel Time (ETT) for the POI to travel to the checkpoint associated with this event, from the POI's last confirmed location, is 5 hours. Based on the ETT, there is a window of time 3304, which represents the possible times at which the POI may leave their required location to attend the event.

In one embodiment, server 102 sends a series of location confirmation messages within the time window 3304, when the POI may leave the required location.

After the time window 3304, the server 102 may continue to perform the location confirmation process for the POI, by sending additional location confirmation messages, to resetting the MDT for the POI. If the POI's location is successfully confirmed via the location confirmation process, the server 102 can remove the POI from the candidate subset, in the event that a person with the same name as the POI attempts to cross a boundary checkpoint associated with the event 3302.

### Estimated Travel Time (ETT)

In one embodiment, the server 102 determines the ETT by using a mapping system, such as Google Maps, to determine the fastest time for the POI reach a checkpoint, using any travel means, from the POI's last confirmed location.

The server 102 applies a realistic travel speed, rather than the Maximum Travel Speed (MTS), when determining the ETT, which is a computation value for estimating worst-case scenario for the travelled distance in a straight line used in the GPV. As an example, prior in the document we have used a value for MTS as 120 km/h. For example, the server 102 makes the assumption that in 2 hours travelling at MTS a POI could reach points in a 240 km radius from their quarantine location.

By comparison for the optimisation of the Monitoring Profiles realistic values need to be used. In reality in an urban area the ETT would correspond to a speed of 40-60 km/h. Using the 2 hours as example a POI could travel 80-120 km on various paths. As the travelling is not in a straight line the checkpoint that a POI can realistically reach would be in an even smaller radius than that.

The first optimisation component is a sequence of location checks—Deterring Checks—that the system will prioritise before and around the time when a POI can attempt a fraudulent trip. This will act as a deterrent.

The second optimisation component is the Reset Message which is one location check performed to reset the MDT. The logic applies also for multiple events. In this case a location check can play dual role such as being part of the “deterrent” for one event and being an MDT re-set for a different event.

The optimised Monitoring Profile will still have to fit within the prescribed parameters of number of messages per period of time (Monitoring Intensity) and maximum gap between checks (MCI).

The logic uses also the concept of Non-essential check which is a normal (random) scheduled check that was not the result of the optimisation process itself. Such messages can be removed to ensure that the number of messages defined by the Monitoring Intensity is not exceeded.

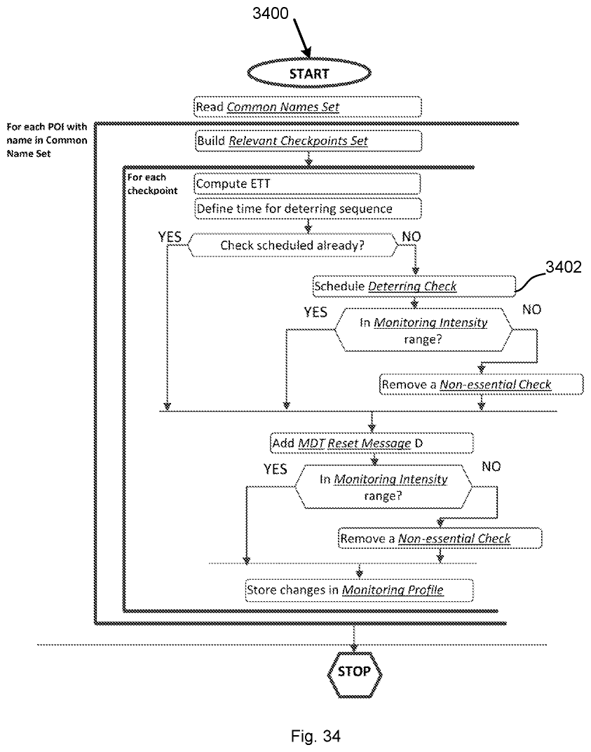

### FIG. 34—Reducing the MDT by Performing the Location Confirmation Process

FIG. 34 is a flowchart illustrating the process 3400 for optimisation of a monitoring profile for a POI, as performed by server 102, according to an embodiment.

In step 3402, additional (deterring) location confirmation checks are around the time when a restricted person will be most likely to attempt fraud. If the candidate subset is empty then the process exits. An empty candidate subset may occur when all restricted persons associated with the boundary checkpoint have had their location confirmed quite recently.

Throughout this specification the word “comprise”, or variations such as “comprises” or “comprising”, will be understood to imply the inclusion of a stated element, integer or step, or group of elements, integers or steps, but not the exclusion of any other element, integer or step, or group of elements, integers or steps.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each of the appended claims.

It will be appreciated by persons skilled in the art that numerous variations and/or modifications may be made to the above-described embodiments, without departing from the broad general scope of the present disclosure. The present embodiments are, therefore, to be considered in all respects as illustrative and not restrictive.
ACK—Admission Check
APV—Amber Path Validation
CP—Checkpoint
CPA—Checkpoint Access area
CPE—Checkpoint Exit area
CPO—Checkpoint Operator, staff at admission point
CRD—Confirmations Reader used on automated checkpoints
CT—Contact Tracking
CTM—Contact Tracking Message
DMA—Decision Making Authority
EA—Enforcing Authority
ECK—Exit Check
ET—Escalation Trigger
GPV—Green Path Validation
HLA—High Level Alert
IRD—Input Reader used on automated checkpoints
LLA—Low Level Alert
MCI—Maximum Check Interval
MNM—Mobile Network Messaging
PAIMCOS—PAndemic IMpact COntrol Solution
POI—Person Of Interest
QCK—Quarantine Check
QM—Quarantine Monitoring
QMM—Quarantine Monitoring Message
VRS—Venue Confirmation Screen used for Contact Tracking

1-24. (canceled)

25. A method for providing anti-spoofing provisions for a sequence of messages sent to a mobile communication device, the method comprising:
sending a first confirmation message to the mobile communication device over a communication means, the first confirmation message comprising a first authentication keyword;
sending a second confirmation message to the mobile communication device over the communication means, the second confirmation message comprising a second authentication keyword and a third authentication keyword; and
sending the third confirmation message to the mobile communication device over the communication means, the third confirmation message comprising a fourth authentication keyword,
wherein the first authentication keyword matches the second authentication keyword, and
wherein the third authentication keyword matches the fourth authentication keyword.

26. The method of claim 25, wherein the first authentication keyword does not match the third authentication keyword.

27. The method of claim 25, further comprising:
establishing a trusted setting associated with a user of the mobile communication device; and
sending the first confirmation message to the mobile communication device when the mobile communication device is in the trusted setting.

28. The method of claim 27,
wherein the first confirmation message comprises an identification code,
wherein the mobile communication device is associated with the user, and
wherein the identification code is uniquely associated with the user.

29. The method of claim 25, wherein the first authentication keyword includes a first sequence number, and the second authentication keyword comprises a second sequence number.

30. The method of claim 29, wherein the second sequence number is incremented with reference to the first sequence number.

31. The method of claim 25, wherein the communication means comprises a short messaging service (SMS).

32. A system for providing anti-spoofing provisions for a sequence of messages sent to a mobile communication device, the system configured to:
send a first confirmation message to the mobile communication device over a communication means, the first confirmation message comprising a first authentication keyword;
send a second confirmation message to the mobile communication device over the communication means, the second confirmation message comprising a second authentication keyword and a third authentication keyword; and
send the third confirmation message to the mobile communication device over the communication means, the third confirmation message comprising a fourth authentication keyword,
wherein the first authentication keyword matches the second authentication keyword, and
wherein the third authentication keyword matches the fourth authentication keyword.

33. A method for authenticating a message of a sequence of messages received by a mobile communication device, the method comprising:
receiving, by the mobile communication device over a communication means, a first confirmation message, the first confirmation message comprising a first authentication keyword;
receiving, by the mobile communication device over the communication means a second confirmation message, the second confirmation message comprising a second authentication keyword and a third authentication keyword;
receiving, by the mobile communication device over the communication means, a third confirmation message, the third confirmation message comprising a fourth authentication keyword; and
comparing the first authentication keyword to the second authentication keyword;
in response to determining that the first authentication keyword matches the second authentication keyword, trusting the second confirmation message; and
in response to determining that the third authentication keyword matches the fourth authentication keyword, trusting the third confirmation message.

34. The method of claim 33, wherein, receiving the first confirmation message comprises, receiving the first confirmation message when the mobile communication device is in a trusted setting.

35. The method of claim 33, further comprising establishing the trusted setting.

36. The method of claim 33, further comprising:
displaying the first confirmation message and the second confirmation message on a display, such that a user of the mobile communication device can visually inspect the first authentication keyword and the second authentication keyword.

37. The method of claim 33, wherein determining that the first authentication keyword matches the second authentication keyword comprises visually comparing the first authentication keyword with the second authentication keyword.

38. The method of claim 33, further comprising,
in response to, determining that the first authentication keyword does not match the second authentication keyword, determining that the second confirmation message is a spoof message.

39. The method of claim 38, further comprising:
in response to determining that the second confirmation message is a spoof message, take steps to re-establish trust in the communication means.
